(19) United States
(12) Patent Application Publication
(10) Pub. No.: US 2026/0028311 A1
(43) Pub. Date: Jan. 29, 2026

(54) RALINEPAG PRODRUGS AND USES THEREOF

(71) Applicant: United Therapeutics Corporation, Silver Spring, MD (US)

(72) Inventors: Hitesh BATRA, Herndon, VA (US); Patrick POISSON, Chapel Hill, NC (US); Liang GUO, Vienna, VA (US); Susovan JANA, Rockville, MD (US); Sri Harsha TUMMALA, Mount Airy, MD (US)

(21) Appl. No.: 19/348,278

(22) Filed: Oct. 2, 2025

Related U.S. Application Data
(63) Continuation of application No. 18/185,844, filed on Mar. 17, 2023.
(60) Provisional application No. 63/321,032, filed on Mar. 17, 2022.

(51) Int. Cl. C07C 271/28; C07C 311/51; C07D 211/32; C07D 295/195; C07D 317/40

(52) U.S. Cl. CPC C07C 271/28; C07C 311/51; C07D 211/32; C07D 295/195; C07D 317/40; C07C 2601/14

(57) ABSTRACT

Described herein are ralinepag prodrugs, as well as pharmaceutical compositions thereof, and methods of use thereof in the treatment of diseases or conditions that would benefit from treatment with a prostacyclin (IP) receptor agonist compound, such as but not limited to pulmonary hypertension (PH) diseases.

### CROSS REFERENCE TO RELATED APPLICATIONS

This application is a continuation of U.S. patent application Ser. No. 18/185,844, filed on Mar. 17, 2023, and claims the benefit of U.S. Provisional Application No. 63/321,032, filed on Mar. 17, 2022, all of which are incorporated herein by reference in its entirety.

### FIELD OF THE INVENTION

Described herein are ralinepag prodrugs which are agonists of the prostacyclin (IP) receptor, as well as pharmaceutical compositions thereof, and methods of use thereof in the treatment of diseases or conditions that would benefit from treatment with ralinepag.

### BACKGROUND

The prostacyclin (IP) receptor is expressed on platelets and on the smooth muscle cells of several tissues, including lung, heart, aorta, liver, kidney, and blood vessels. Activation of the IP receptor results in increased cellular cyclic adenosine monophosphate (CAMP) followed by vasodilation in arteries and inhibition of aggregation in platelets. Improved hemodynamics, exercise capacity, and survival have been clearly demonstrated for PGI₂ replacement therapies for the treatment of, for example, pulmonary hypertension (PH) and pulmonary arterial hypertension (PAH).

### SUMMARY

Disclosed herein is a compound of Formula (I), or a pharmaceutically acceptable salt, solvate, or stereoisomer thereof:

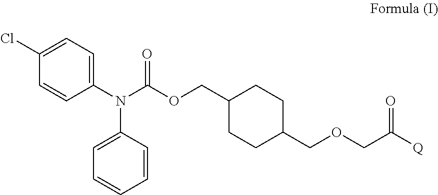

wherein:
Q is —NR⁶—S(═O)R⁷;
R⁶ is H, C₁-C₂₄alkyl, C₁-C₂₄ haloalkyl, C₂-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
R⁷ is C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
L² is absent, C₁-C₆ alkylene, C₁-C₆ heteroalkylene, C₂-C₆ alkenylene, or C₂-C₆ alkynylene, each of which is optionally substituted with one or more R^{7a},
or Q is —OR⁸;
R⁸ is —CH₂CH₂CH₃, —CH(CH₃)₂, —CH₂CH₂CH₂CH₃, —CH₂CH(CH₃)₂, C₅-C₂₄ alkyl, C₁-C₂₄ haloalkyl, C₁-C₂₄ heteroalkyl, C₂-C₂₄ alkenyl, C₂-C₂₄ alkynyl, -L³-O—P(═O)(OH)₂, -L³-cycloalkyl, -L³-heterocycloalkyl, or -L³-aryl, wherein each of the alkyl, heteroalkyl, alkenyl, alkynyl, cycloalkyl, heterocycloalkyl, or aryl is optionally substituted with one or more R^{8a};
L³ is absent, C₁-C₆ alkylene, C₁-C₆ heteroalkylene, C₂-C₂₄alkenylene, or C₂-C₂₄ alkynylene, each of which is optionally substituted with one or more R^{8a};
or Q is —NR⁴R⁵;
R⁴ is H, C₁-C₂₄ alkyl, C₁-C₂₄ haloalkyl, C₁-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a};
R⁵ is C₁-C₂₄ alkyl, C₁-C₂₄ haloalkyl, C₂-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a}; or
R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a};
L⁵ is absent, C₁-C₆ alkylene, C₁-C₆ heteroalkylene, C₂-C₂₄ alkenylene, or C₂-C₂₄ alkynylene, each of which is optionally substituted with one or more R^{5a};
R^{5a} and R^{7a} are each independently selected from halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
R^{8a} is selected from halogen, —CN, —NO₂, —OH, —OR^{a}, oxo, —OC(═O)R^{a}, —SH, —SRₐ, —S(═O)R^{a}, —S(═O)₂R^{a}, —S(═O)₂OR^{b}, —S(═O)—NR^{c}R^{d}, —NR^{c}R^{d}, —NR^{b}C(═O)R^{a}, —NR^{b}S(═O)₂R^{a}, —C(═O)R^{a}, —C(═O)OR^{b}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
each R^{a} is independently C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl (cycloalkyl),
C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₁-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R; or
R^{c} and R^{d} are taken together with the atom to which they are attached to form a heterocycloalkyl optionally substituted with one or more R; and
each R is independently halogen —CN, —OH, oxo, —OC₁-C₆alkyl, —S(═O)C₁-C₆alkyl, —S(═O)₂C₁-C₆alkyl, —S(═O)₂NH₂, —S(—O)₂NHC₁-C₆alkyl, —S(═O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl); —C(═O)NHC₁-C₆alkyl, C₁-C₆alkyl, C₁-C₆haloalkyl, or C₁-C₆heteroalkyl;
or Q is: 1-carboxyethylamino, 1-carboxy-4-guanidinobutylamino, 3-amino-1-carboxy-3-oxopropylamino, 1,2-dicarboxyethylamino, 1-carboxy-2-mercaptoethylamino, 4-amino-1-carboxy-4-oxobutylamino, 3-carboxy-1-carboxylatepropylamino, 1-carboxy-2-(1H-imidazol-4-yl)ethylamino, 1-carboxy-2-methylbutylamino, 1-carboxy-3-methylbutylamino, 5-amino-1-carboxypentylamino, 1-carboxy-3-(methylthio)propylamino, 1-carboxy-2-phenylethylamino, 2-carboxypyrrolidin-1-yl, 1-carboxy-2-hydroxyethylamino, 1-carboxy-2-hydroxypropylamino, 1-carboxy-2-(1H-indol-3-yl)ethylamino, 1-carboxy-2-(4-hydroxyphenyl)ethylamino and 1-carboxy-2-methylpropylamino.

Also described herein is a pharmaceutical composition, comprising a compound described herein, or a pharmaceutically acceptable salt or solvate thereof, and a pharmaceutically acceptable carrier or excipient.

Also described herein is a method of treating pulmonary arterial hypertension (PAH) in a subject in need thereof, comprising administering to the subject a compound described herein, or a pharmaceutically acceptable salt or solvate thereof, or a pharmaceutical composition described herein.

Also described herein is a method of modulating a prostacyclin (PGI2) receptor in a subject in need thereof, comprising administering to the subject a compound described herein, or a pharmaceutically acceptable salt or solvate thereof, or a pharmaceutical composition described herein.

Also described herein is a method of treating a disease or condition associated with a prostacyclin (PGI2) receptor in a subject in need thereof, comprising administering to the subject a compound described herein, or a pharmaceutically acceptable salt or solvate thereof, or a pharmaceutical composition described herein.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION

Disclosed herein are compounds that are suitable for use as prostacyclin (IP) receptor agonist and methods of using the same. In one aspect, described herein are prodrugs of Ralinepag and methods of using the same. Ralinepag is an oral IP receptor agonist. Ralinepag is also named 2-(((1r,4r)-4-(((4-chlorophenyl)(phenyl)carbamoyloxy)methyl)cyclohexyl)methoxy)acetic acid and has the following structure:

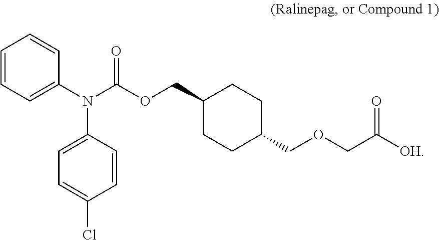 [SMILES: O=C(O)COC[C@H]1CC[C@H](COC(=O)N(c2ccccc2)c2ccc(Cl)cc2)CC1]

### Definitions

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:
“oxo” refers to ═O.
“Carboxyl” refers to —COOH.
“Cyano” refers to —CN.
“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-₁₀alkyl. In some embodiments, the alkyl is a C₁-₆alkyl. In some embodiments, the alkyl is a C₁-₅alkyl, In some embodiments, the alkyl is a C₁-₄alkyl. In some embodiments, the alkyl is a C₁-₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.
“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH—CH═CH₂), isopropenyl [—C(CH₃)═CH₂], butenyl, 1,3-butadienyl and the like. Whenever it appears herein, a numerical range such as “C₂-C₆ alkenyl” or “C₂-₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.
“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C-alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.
“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —CO₂H, CO₂Me, —OH, —OMe, —NH₂, or —NO₂. In some embodiments, the alkylene is optionally substituted with halogen, —CN, —OH, or —OMe. In some embodiments, the alkylene is optionally substituted with halogen.
“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —CO₂H, —CO₂Me, —OH, —OMe, —NH₂, or —NO₂. In some embodiments, the alkoxy is optionally substituted with halogen, —CN, —OH, or —OMe. In some embodiments, the alkoxy is optionally substituted with halogen.
“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —CO₂H, —CO₂Me, —CF₃—OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.
“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₅ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl. cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —CO₂H, CO₂Me, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.
“Halo” or “halogen” refers to bromo, chloro, fluoro or iodo. In some embodiments, halogen is fluoro or chloro. In some embodiments, halogen is fluoro
“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.
“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.
“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.
“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a CI-Ce heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.
“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems, and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₇ heterocycloalkenyl), from two to five carbon atoms (C₂-C₄ fully saturated heterocycloalkyl or C₂-C₄ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxothiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, —COOMe, —CF₃, —OH, —OMe, —NH₂, or —NO₂. In some embodiments, the heterocycloalkyl is optionally substituted with halogen, methyl, ethyl, —CN, —CF, —OH, or —OMe. In some embodiments, the heterocycloalkyl is optionally substituted with halogen.
“Heteroaryl” refers to a 5- to 14-membered ring system radical comprising one to thirteen carbon atoms, one to six heteroatoms selected from the group consisting of nitrogen, oxygen. phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —CO₂H, CO₂Me, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.
The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.
The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.
An “effective amount” or “therapeutically effective amount” refers to an amount of a compound administered to a mammalian subject, either as a single dose or as part of a series of doses, which is effective to produce a desired therapeutic effect.
The terms “treat,” “treating” or “treatment” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.
The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.
The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), and topical administration. Those of skill in the art are familiar with administration techniques that can be employed with the compounds and methods described herein. In some embodiments, the compounds and compositions described herein are administered orally.
The terms “co-administration” or the like, as used herein, are meant to encompass administration of the selected therapeutic agents to a single patient, and are intended to include treatment regimens in which the agents are administered by the same or different route of administration or at the same or different time.

### Compounds

Described herein are compounds, or a pharmaceutically acceptable salt, solvate, or stereoisomer thereof useful in the treatment of a disease or disorder associated with pulmonary hypertension (PH), pulmonary vascular resistance (PVR), pulmonary arterial hypertension (PAH), pulmonary hypertension associated with interstitial lung disease (PH-ILD), or a combination thereof.

Disclosed herein is a compound of Formula (I), or a pharmaceutically acceptable salt, solvate, or stereoisomer thereof:

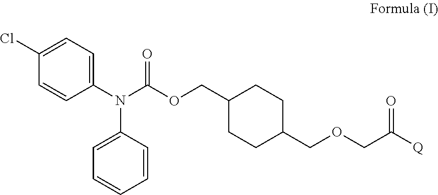

wherein:
Q is —NR₆—S(═O)₂R⁷;
   R⁶ is H, C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a}.
   R⁷ is C₁-C₂₄alkyl, C₁-C₂₄ haloalkyl, C₁-C₂₄ heteroalkyl, C₂-C₂₄ alkenyl, C₂-C₂₁ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
   L² is absent, C₁-C₆ alkylene, C₁-C₆ heteroalkylene, C₂-C₆ alkenylene, or C₂-C₆ alkynylene, each of which is optionally substituted with one or more R^{7a};
or Q is —OR⁸;
   R⁸ is —CH₂CH₂CH₃, —CH(CH₃)₂, —CH₂CH₂CH₂CH₃, —CH—CH(CH₃)₂, C₅-C₂₄ alkyl, C₂-C₂₄ haloalkyl, C₁-C₂₄ heteroalkyl, C₂-C₂₄ alkenyl, C₂-C₂₄ alkynyl, -L³-O—P(═O)(OH)₂, -L³-cycloalkyl, -L³-heterocycloalkyl, or -L³-aryl, wherein each of the alkyl, heteroalkyl, alkenyl, alkynyl, cycloalkyl, heterocycloalkyl, or aryl is optionally substituted with one or more R^{8a};
   L³ is absent, C₁-C₆ alkylene, C₁-C₆ heteroalkylene, C₂-C₆ alkenylene, or C₂-C₂₄ alkynylene, each of which is optionally substituted with one or more R^{8a};
or Q is —NR⁴R⁵;
   R⁴ is H, C₁-C₂₄ alkyl, C₁-C₂₄ haloalkyl, C₁-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a};
   R⁵ is C₁-C₂₄ alkyl, C₁-C₂₄ haloalkyl, C₁-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a}; or
   R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a};
   L⁵ is absent, C₁-C₆ alkylene, C₁-C₆ heteroalkylene, C₂-C₆ alkenylene, or C₂-C₂₄ alkynylene, each of which is optionally substituted with one or more R^{5a};
   R^{5a} and R^{7a} are each independently selected from halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ heteroalkyl, C₁-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
   R^{8a} is selected from halogen, —CN, —NO₂, —OH, —OR^{a}, oxo, —OC(═O)R^{a}, —SH, —SR^{a}, —S(═O)R^{a}, —S(═O)₂R^{a}, —S(═O)₂OR^{b}, —S(═O)₂NR^{c}R^{d}, —NR^{c}R^{d}, —NR^{b}C, (═O)R^{a}, —NR^{b}S(═O)₂R^{a}, —C(═O)R^{a}, —C(═O)OR^{b}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
   each R^{a} is independently C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl heteroaryl); wherein each alkyl, alkenyl, alkynyl, cycloalkyl, heterocycloalkyl, aryl, and heteroaryl is independently optionally substituted with one or more R;
   each R^{b} is independently hydrogen, C₁-C₆alkyl, C₁-C₆haloalkyl, C₁-C₆heteroalkyl, C₂-C₆alkenyl, C₁-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
   each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl heteroaryl); wherein each alkyl, alkenyl, alkynyl, cycloalkyl, heterocycloalkyl, aryl, and heteroaryl is independently optionally substituted with one or more R; or
   R^{c} and R^{d} are taken together with the atom to which they are attached to form a heterocycloalkyl optionally substituted with one or more R; and
   each R is independently halogen, —CN, —OH, oxo, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆heteroalkyl;
or Q is: 1-carboxyethylamino, 1-carboxy-4-guanidinobutylamino, 3-amino-1-carboxy-3-oxopropylamino, 1,2-dicarboxyethylamino, 1-carboxy-2-mercaptoethylamino, 4-amino-1-carboxy-4-oxobutylamino, 3-carboxy-1-carboxylatepropylamino, 1-carboxy-2-(1H-imidazol-4-yl)ethylamino, 1-carboxy-2-methylbutylamino, 1-carboxy-3-methylbutylamino, 5-amino-1-carboxypentylamino, 1-carboxy-3-(methylthio)propylamino, 1-carboxy-2-phenylethylamino, 2-carboxypyrrolidin-1-yl, 1-carboxy-2-hydroxyethylamino, 1-carboxy-2-hydroxypropylamino, 1-carboxy-2-(1H-indol-3-ylethylamino, 1-carboxy-2-(4-hydroxyphenyl)ethylamino and 1-carboxy-2-methylpropylamino.

Disclosed herein is a compound of Formula (V), or a pharmaceutically acceptable salt, solvate, or stereoisomer thereof:

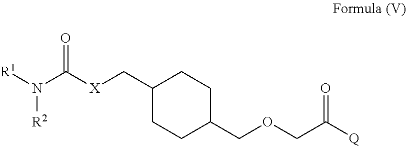

wherein:
R¹ and R² are each independently selected from: phenyl and monocyclic heteroaryl; wherein the phenyl and heteroaryl are each optionally substituted with one or two substituents selected from: C₁-C₆ alkoxy, C₁-C₆ alkyl, aryl, C₁-C₆ haloalkoxy, C₁-C₆ haloalkyl, C₁-C₆ hydroxyalkyl, C₁-C₆ aminoalkyl, C₁-C₆ heteroalkyl, and halogen;
X is O or NR³;
R³ is selected from H and C₁-C₆ alkyl; and
Q is —NR⁶—S(═O)₂R⁷;
   R⁶ is H, C₁-C₂₄ alkyl, C₁-C₂₄ haloalkyl, C₁-C₂₄ heteroalkyl, C₂-C₂₄ analkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
   R⁷ is C₁-C₂₄ alkyl, C₁-C₂₄ haloalkyl, C₂-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
   L² is absent C₁-C₆ alkylene, C₁-C₆ heteroalkylene, C₂-C₆ alkenylene, or C₂-C₆ alkynylene, each of which is optionally substituted with one or more R^{7a};
or Q is —OR⁸;
   R⁸ is —CH₂CH₂CH₃, —CH(CH₃)₂, —CH₂CH₂CH₂CH₃, —CH₂CH(CH₃)₂, C₅-C₂₄ alkyl, C₁-C₂₄ haloalkyl, C₁-C₂₄ heteroalkyl, C₂-C₂₄ alkenyl, C₂-C₂₄ alkynyl, -L³-O—P(═O)(OH)₂, -L³-cycloalkyl, -L³-heterocycloalkyl, or -L³-aryl, wherein each of the alkyl, heteroalkyl, alkenyl, alkynyl, cycloalkyl, heterocycloalkyl, or aryl is optionally substituted with one or more R^{8a};
   L³ is absent, C₁-C₆ alkylene, C₁-C₆ heteroalkylene, C₂-C₆ alkenylene, or C₂-C₂₄ alkynylene, each of which is optionally substituted with one or more R^{8a};
or Q is —NR⁴R⁵;
   R⁴ is H, C₁-C₂₄ alkyl, C₁-C₂₄ haloalkyl, C₂-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a};
   R⁵ is C₁-C₂₄ alkyl, C₁-C₂₄ haloalkyl, C₁-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a}; or
   R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a};
   L⁵ is absent, C₁-C₆ alkylene, C₁-C₆ heteroalkylene, C₂-C₆ alkenylene, or C₂-C₂₄ alkynylene, each of which is optionally substituted with one or more R^{5a};
   R^{5a} and R^{7a} are each independently selected from halogen, —CN, —NO₂, —OH, —OR^{a}, oxo, —OC(═O)R^{a}, —SH, —SR^{a}, —S(═O)R^{a}, —S(═O)₂R^{a}, —S(═O)NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆alkyl, C₁-C₆ haloalkyl, C₁-C₆ heteroalkyl, C₂-C₆, alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
   R^{8a} is selected from halogen, —CN, —NO₂, —OH, —OR^{a}, oxo, —OC(═O)R^{a}, —SH, —SR^{a}, —S(═O)R^{a}, —S(═O)₂R^{a}, —S(═O)₂OR^{b}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{b}, —C(═O)NR^{c}R^{d}, C₁-C₆alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
   each R^{a} is independently C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl (heteroaryl); wherein each alkyl, alkenyl, alkynyl, cycloalkyl, heterocycloalkyl, aryl, and heteroaryl is independently optionally substituted with one or more R;
   each R^{b}is independently hydrogen, C₁-C₆alkyl, C₁-C₆haloalkyl, C₁-C₆eteroalkyl, C₂-C₆alkenyl, C₁-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
   each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R; or
   R^{c} and R^{d} are taken together with the atom to which they are attached to form a heterocycloalkyl optionally substituted with one or more R; and
   each R is independently halogen, —CN, —OH, oxo, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(—O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆heteroalkyl;
or Q is selected from: 1-carboxyethylamino, 1-carboxy-4-guanidinobutylamino, 3-amino-1-carboxy-3-oxopropylamino, 1,2-dicarboxyethylamino, 1-carboxy-2-mercaptoethy lamino, 4-amino-1-carboxy-4-oxobutylamino, 3-carboxy-1-carboxylatepropylamino, 1-carboxy-2-(1H-amino-4-carboxypentylamino, 1-carboxy-3-(methylthio)propylamino, 1-carboxy-2-phenylethylamino, 2-carboxypyrrolidin-1-yl, 1-carboxy-2-hydroxyethylamino, 1-carboxy-2-hydroxypropylamino, 1-carboxy-2-(1H-indol-3-yl)ethylamino, 1-carboxy-2-(4-hydroxyphenyl)ethylamino and 1-carboxy-2-methylpropylamino.

In some embodiments, the compound or a pharmaceutically acceptable salt or solvate thereof, has a structure of Formula (IIIa), or a pharmaceutically acceptable salt or solvate thereof:

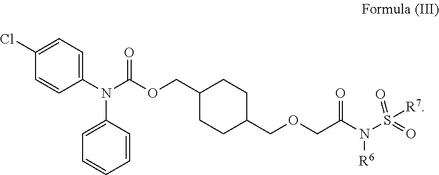

In some embodiments, the compound or a pharmaceutically acceptable salt or solvate thereof, has a structure of Formula (IIIa):

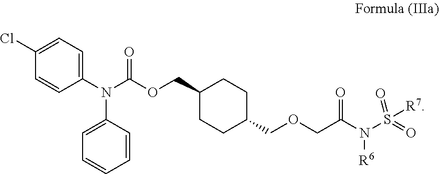

R⁶ can be any suitable functional group known by one of skill in the art. In some embodiments. R⁶ is H, C₁-C₂₄alkyl, C₁-C₂₄haloalkyl, C₁-C₂₄heteroalkyl, C₂-C₂₄alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a}. In some embodiments, R⁶ is H, C¹-C₂₄ alkyl, C₁C₂₄ haloalkyl, C₁-C₂₄ heteroalkyl, or C₁-C₂₄ alkenyl. In some embodiments, R⁶ is H, C₁-C₂₄alkyl, or C₁-C₂₄ haloalkyl. In some embodiments, R⁶ is H or C₁-C₂₄ alkyl. In some embodiments, R⁶ is H or C₁-C₁₂ alkyl. In some embodiments, R⁶ is H or C₁-C₆ alkyl. In some embodiments. R⁶ is H.

R⁷ can be any suitable functional group known by one of skill in the art. In some embodiments, R⁷ is C₁-C₂₄ alkyl, C₁-C₂₄ haloalkyl, C₁-C₂₄ heteroalkyl, C₂-C₂₄ alkenyl, C₂-C₂₄ alkynyl, —L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted. In some embodiments, R⁷ is C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a}. In some embodiments, R⁷ is C₁-C₁₂alkyl, C₁-C₁₂ haloalkyl, C₁-C₁₂ heteroalkyl, C₂-C₁₂ alkenyl, C₂-C₁₂ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted. In some embodiments, R⁷ is C₁-C₂₄alkyl, C₁-C₂₄ haloalkyl, C₁-C₂₄ heteroalkyl, or C₂-C₂₄ alkenyl. In some embodiments, R⁷ is C₁-C₂₄ alkyl, C₁-C₂₄ haloalkyl, or C₁-C₂₄ heteroalkyl, wherein each of the alkyl or heteroalkyl is optionally substituted with one or more R^{7a}. In some embodiments, R⁷ is C₁-C₁₂ alkyl, C₁-C₁₂ haloalkyl, C₁-C₁₂ heteroalkyl, or C₂-C₁₂ alkenyl, wherein each of which is optionally substituted. In some embodiments, R⁷ is C₁-C₁₂ alkyl, C₁-C₁₂ haloalkyl, or C₁-C₁₂ heteroalkyl, wherein each of which is optionally substituted. In some embodiments, R⁷ is C₁-C₆ alkyl, C₁-C₆ haloalkyl, or C₁-C₆ heteroalkyl, wherein each of which is optionally substituted. In some embodiments, R⁷ is optionally substituted C₁-C₆ alkyl. In some embodiments, R⁷ is C₁-C₁₂ alkyl. In some embodiments, R⁷ is optionally substituted C₁-C₄ alkyl. In some embodiments, R⁷ is C₁-C₆ alkyl. In some embodiments, R⁷ is optionally substititued C₁-C₄ alkyl. In some embodiments, R⁷ is C₁-C₄ alkyl. In some embodiments, wherein R⁷ is optionally substituted —CH₃, —CH₂CH₃, —CH₂CH₂CH₃, —CH(CH₃)₂, —CH₂CH₂CH₂CH₃, —CH₂CH(CH₃)₂, —CH₂CH₂CH₂CH₂CH₃, or —CH₂CH₂CH₂CH₂CH₂CH₃. In some embodiments, R⁷ is —CH₃, —CH₂CH₃, —CH₂CH₂CH₃, —CH(CH₃)₂, —CH₂CH₂CH₂CH, —CH₂CH(CH₃)₂, —CH₂CH₂CH₂CH₂CH₃, or —CH₂CH₂CH₂CH₂CH₂CH₃. In some embodiments, wherein R⁷ is optionally substituted —CH₃, —CH₂CH₃, —CH₂CH₂CH₃, —CH(CH₃)₂, —CH₂CH₂CH₂CH₃, —CH₂CH(CH₃)₂. In some embodiments, R⁷ is —CH₃, —CH₂CH₃, —CH₂CH₂CH₃, —CH(CH₃)₂, —CH₂CH₂CH₂CH₃, —CH₂CH(CH₃)₂. In some embodiments, R⁷ is methyl,

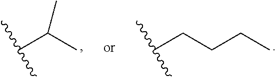

In some embodiments, R⁷ is methyl.

In some embodiments, R⁷ is -L²-cycloalkyl, -L⁷-heterocycloalkyl, -L²-aryl, or -L²-heteroaryl, wherein each of the cycloalkyl, heterocycloalkyl, aryl or heteroaryl is optionally substituted with one or more R^{7a}; and R^{2a} is halogen, —CN, —OH, —OR^{a}, oxo, —OC(═O)R^{a}, —SH, —SR^{a}, —S(═O)R^{a}, —S(═O)₂R^{a}, —S(═O)NR^{c}R^{d}, —NR^{c}R^{d}, —C(═O)R^{a}, —C(═O)OR^{a}, C₁-C₆ alkyl, C₁-C₆ haloalkyl, or C₁-C₆ heteroalkyl, wherein each of the alkyl or heteroalkyl is optionally substituted with one or more R. In some embodiments, R⁷ is -L²-cycloalkyl, -L²-heterocycloalkyl, -L²-aryl, or -L²-heteroaryl, wherein each of the cycloalkyl, heterocycloalkyl, aryl or heteroaryl is optionally substituted with one or more R^{7a}; and R⁷ is halogen, —OH, —OR^{a}, oxo, C₁-C₆ alkyl, or C₁-C₆ haloalkyl, wherein each of the alkyl or heteroalkyl is optionally substituted with one or more R. In some embodiments, R⁷ is -L²-cycloalkyl, -L²-heterocycloalkyl, -L²-aryl, or -L²-heteroaryl, wherein each of the cycloalkyl, heterocycloalkyl, aryl or heteroaryl is optionally substituted with one or more R^{7a}, and R^{7a} is halogen, —OH, —OR^{a}, oxo, C₁-C₆ alkyl, or C₁-C₆ haloalkyl. In some embodiments, R⁷ is -L²-heterocycloalkyl, -L²-aryl, or -L²-heteroaryl, wherein each of the heterocycloalkyl, aryl or heteroaryl is optionally substituted with one or more R^{7a}; and R^{7a} is halogen, —OH, —OR^{a}, oxo, C₁-C₆ alkyl, or C₁-C₆ haloalkyl. In some embodiments, R⁷ is -L²-heterocycloalkyl, -L²-aryl, or -L²-heteroaryl, wherein each of the heterocycloalkyl, aryl or heteroaryl is optionally substituted with one or more R^{7a}; and R^{7a} is halogen, —OH, —OR^{a}, oxo, C₁-C₆ alkyl, or C₁-C₆ haloalkyl, and R^{a} is methyl or ethyl. In some embodiments, R⁷ is -L²-heterocycloalkyl, -L²-aryl, or -L²-heteroaryl, wherein each of the heterocycloalkyl, aryl or heteroaryl is optionally substituted with one or more R^{7a}; and R^{7a} is halogen, —OH, —OR^{a}, oxo, C₁-C₃ alkyl, or C₁-C₃ haloalkyl. In some embodiments, R⁷ is -L²-thiochromanyl, -L²-phenyl, L²-naphthyl, -L²-thiophen-2-yl, or -L²-benzothiazolyl, wherein each of the thiochromanyl, phenyl, naphthyl, thiophen-2-yl, or benzothiazolyl is optionally substituted with one or more R^{7a}; and R^{7a} is halogen, —OH, —OR₃, oxo, C₁-C₃ alkyl, or C₁-C₃ haloalkyl. In some embodiments, R⁷ is -L²-thiochromanyl, -L²-phenyl, L²-naphthyl, -L²-thiophen-2-yl, or -L²-benzothiazolyl, wherein each of the thiochromanyl, phenyl, naphthyl, thiophen-2-yl, or benzothiazolyl is optionally substituted with one or more R^{7a}, and R^{7a} is chloro, —OH, —OCH₃, —OCH₂CH₃, oxo, —CH₃, or —CF₃. In some embodiments, R⁷ is -L²-phenyl or -L²-naphthyl, wherein each of the phenyl or naphthyl is optionally substituted with one or more R^{7a}, L² is absent, —CH₂—, or —CH₂CH₂—; and R^{7a} is halogen, oxo, C₁-C₆ alkyl, C₁-C₆ haloalkyl, or C₁-C₆ alkoxy. In some embodiments, R⁷ is -L²-phenyl or -L²-naphthyl, wherein each of the phenyl or naphthyl is optionally substituted. In some embodiments, R⁷ is -L²-phenyl or -L²-naphthyl, wherein each of the phenyl or naphthyl is optionally substituted with one or more R^{7a}, L²is absent, and R^{7a} is halogen, oxo, C₁-C₆ alkyl, C₁-C₆ haloalkyl, or C₁-C₆ alkoxy. In some embodiments, R⁷ is -phenyl or -naphthyl, wherein each of the phenyl or naphthyl is optionally substituted with one or more R^{7a}, and R^{7a} is halogen, oxo, C₁-C₆ alkyl, C₁-C₆ haloalkyl, or C₁-C₆ alkoxy. In some embodiments, R⁷ is -phenyl or -naphthyl, wherein each of the phenyl or naphthyl is optionally substituted. In some embodiments, R⁷ is -phenyl or -naphthyl, wherein each of the phenyl or naphthyl is optionally substituted with one or more R^{7a}, and R^{7a} is chloro, oxo, —CH₃, —CF₃, or —OCH₃. In some embodiments, R⁷ is -L²-heteroaryl, wherein the heteroaryl is bicyclic and is optionally substituted with one or more R^{7a}. In some embodiments, R⁷ is -L²-heteroaryl, wherein the heteroaryl is monocyclic and is optionally substituted with one or more R^{7a}. In some embodiments, R⁷ is -L²-cycloalkyl, wherein the cycloalkyl is bicyclic and is optionally substituted with one or more R^{7a}. In some embodiments, R⁷ is -L²-cycloalkyl, wherein the cycloalkyl is monocyclic and is optionally substituted with one or more R^{7a}. In some embodiments, R⁷ is -L²-heterocycloalkyl, wherein the heterocycloalkyl is bicyclic and is optionally substituted with one or more R^{7a}. In some embodiments, R⁷ is -L²-heterocycloalkyl, wherein the heterocycloalkyl is monocyclic and is optionally substituted with one or more R^{7a}. In some embodiments, R⁷ is -L²-naphthyl, wherein the naphthyl is optionally substituted with one or more R^{7a}.

L² can be any suitable linker known by one of skill in the art. In some embodiments, L² is absent, C₁-C₆alkylene, C₁-C₆ heteroalkylene, C₂-C₆ alkenylene, or C₂-C₆ alkynylene, each of which is optionally substituted with one or more R^{7a}. In some embodiments, L²is absent, —CH₂—, or —CH₂CH₂—. In some embodiments, L² is absent. C₁-C₆ alkylene, C₁-C₆ heteroalkylene, C₂-C₆ alkenylene, or C₂-C₆ alkynylene, each of which is optionally substituted. In some embodiments, L² is absent, or C₁-C₆ alkylene. In some embodiments, L² is absent. In some embodiments, L² is C₁-C₆ alkylene or C₁-C₆ heteroalkylene, each of which is optionally substituted. In some embodiments, L²is C₁-C₆ alkylene. In some embodiments, L²is C₁-C₃ alkylene. In some embodiments, L² is C₁-C₃ heteroalkylene,

R^{7a} can be any suitable functional group known by one of skill in the art. In some embodiments, R^{7a} is halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O) NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R. In some embodiments, R^{7a} is cycloalkyl, heterocycloalkyl, aryl, or heteroaryl, wherein each of the cycloalkyl, heterocycloalkyl, aryl, or heteroaryl, is optionally substituted with one or more R. In some embodiments, R^{7a} is halogen, —CN, —NO₂, —OH, —OR^{a}, oxo, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{c}C(═O)OR^{b}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ hydroxyalkyl, C₁-C₆ aminoalkyl, or C₁-C₆ heteroalkyl. In some embodiments, R^{7a} is halogen, —OR^{a}, oxo, —C(═O)OR^{a}, C₁-C₆alkyl, or C₁-C₆ heteroalkyl. In some embodiments, R^{7a} is halogen, —OC₁-C₃alkyl, oxo, —C(═O)OC₁-C₃alkyl, C₁-C₃ alkyl, or C₁-C₃ heteroalkyl. In some embodiments, R^{7a} is halogen, —OC₁-C₃alkyl, oxo, C₁-C₃ alkyl, or C₁-C₃ heteroalkyl. In some embodiments, R^{7a} is halogen,—OC₁-C₃alkyl, oxo, C₁-C₃ alkyl, or C₁-C₃ haloalkyl. In some embodiments, R^{7a} is chloro, —OCH₃, —OCH₂CH₃, oxo, —CH₃, or —CF₃.

In some embodiments, R⁷ is optionally substituted aryl or optionally substituted heteroaryl. In some embodiments, R⁷ is aryl or heteroaryl, wherein each of the aryl or heteroaryl is optionally substituted with one or more R. In some embodiments, R⁷ is

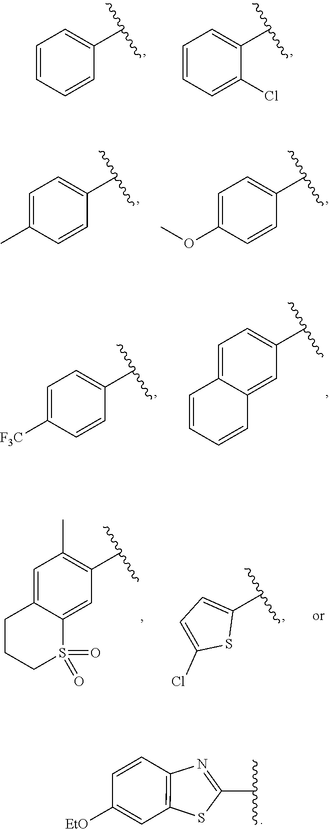

In some embodiments, R⁶ is hydrogen or C₁-C₆ alkyl, and R⁷ is C₁-C₂₄ alkyl, -L²-cycloalkyl, -L²-aryl, or -L²-heteroaryl, wherein each of the alkyl, cycloalkyl, aryl, or heteroaryl is optionally substituted with halogen, —OH, oxo, C₁-C₆ alkyl, C₁-C₆ haloalkyl, or —OC₁-C₆ alkyl. In some embodiments, R⁶ is hyodrgen or C₁-C₃ alkyl, and R⁷ is C₁-C₁₂ alkyl, -L²-cycloalkyl, -L-aryl, or -L²-heteroaryl, optionally substituted. In some embodiments, R⁶ is hydrogen and R⁷ is C₁-C₆ alkyl, -L²-aryl, or -L²-heteroaryl, wherein each of the alkyl, aryl, or heteroaryl is optionally substituted with halogen, oxo, C₁-C₆ alkyl, C₁-C₆ haloalkyl, or —OC₁-C₆ alkyl. In some embodiments, R⁶ is hydrogen and R⁷ is —CH₃, —CH₂CH₃, —CH₂CH₂CH₃, —CH(CH₃)₂, —CH₂CH₂CH₂CH₃, —CH₂CH(CH₃)₂. In some embodiments, ⁶ is hydrogen and R⁷ is -L²-thiochromanyl, -L²-phenyl, L²-naphthyl, -L²-thiophen-2-yl, or -L²-benzothiazolyl, wherein each of the thiochromanyl, phenyl, naphthyl, thiophen-2-yl, or benzothiazolyl is optionally substituted with one or more R^{7a}; and R^{7a} is chloro, —OH, —OCH₃, —OCH₂CH₃, oxo, —CH₃, or —CF₃. In some embodiments, R⁶ is hydrogen and R⁷ is -phenyl or -naphthyl, wherein each of the phenyl or naphthyl is optionally substituted with one or more R^{7a}, and R^{7a} is chloro, oxo, —CH₃, —CF₃, or —OCH₃. In some embodiments, R⁶ is hydrogen and R⁷ is methyl. In some embodiments, the structure

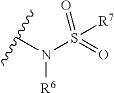

is

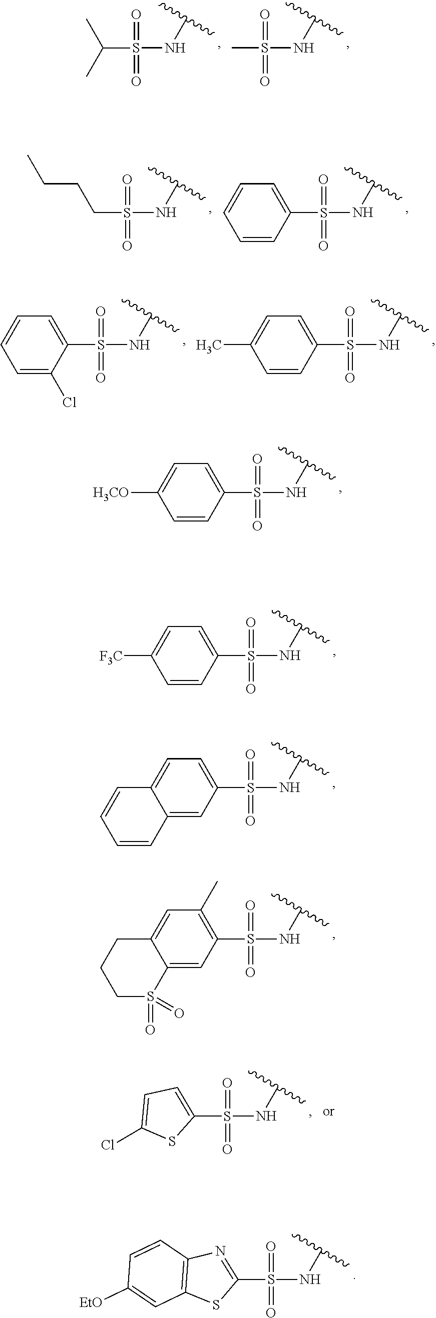

In some embodiments, the structure

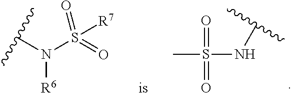

In some embodiments, the compound or a pharmaceutically acceptable salt or solvate thereof, has a structure of Formula (II)

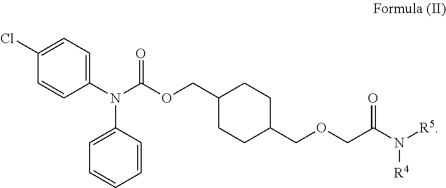

In some embodiments, the compound or a pharmaceutically acceptable salt or solvate thereof, has a structure of Formula (IIa):

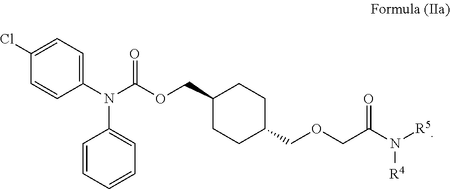

R⁴ can be any suitable functional group known by one of skill in the art. In some embodiments, R⁴ is H, C₁-C₂₄ alkyl, C₁-C₂₄ haloalkyl, C₁-C₂₄ heteroalkyl, C₁-C₂₄ alkenyl, C₂-C₂₄ alkynyl, -L⁵-cycloalkyl, -L⁵-heterocycloalkyl, -L⁵-aryl, or -L⁵-heteroaryl, wherein each of the alkyl, heteroalkyl, alkenyl, alkynyl, cycloalkyl. In some embodiments, R⁴ is H, C₁-C₂₄ alkyl, C₁-C₂₄ haloalkyl, or C₁-C₂₄ heteroalkyl. In some embodiments, R⁴ is H, C₁-C₁₂ alkyl, C₁-C₁₂ haloalkyl, or C₁-C₁₂ heteroalkyl. In some embodiments, R⁴ is H, C₁-C₆ alkyl, C₁-C₆ haloalkyl, or C₁-C₆ heteroalkyl. In some embodiments, R⁴ is H or C₁-C₁₂ alkyl. In some embodiments, R⁴ is H or C₁-C₆ alkyl. In some embodiments, R⁴ is H. In some embodiments, R⁴ is C₁-C₆ alkyl. In some embodiments, R⁴ is C₁-C₃ alkyl. In some embodiments, R⁴ is —CH₃.

R⁵ can be any suitable functional group known by one of skill in the art. In some embodiments, R⁵ is C₁-C₂₄ alkyl, C₁-C₂₄ haloalkyl, C₁-C₂₄ heteroalkyl, C₂-C₂₄ alkenyl, C²-C²⁴ alkynyl, -L²-cycloalkyl, -L²-heterocycloalkyl, -L⁵-aryl, or -L⁵-heteroaryl, wherein each of the alkyl, heteroalkyl, alkenyl, alkynyl, cycloalkyl, heterocycloalkyl, aryl or heteroaryl is optionally substituted with one or more R^{5a}. In some embodiments, R⁵ is C₁-C₂₄ alkyl, C₁-C₂₄ heteroalkyl, -L⁵-aryl, or -L⁵-heteroaryl, wherein each of the alkyl, heteroalkyl, aryl, or heteroaryl is optionally substituted with one or more R^{5a}. In some embodiments, R⁵ is C₁-C₁₂ alkyl, C₁-C₁₂ haloalkyl, C₁-C₁₂ hydroxyalkyl, C₁-C₁₂ aminoalkyl. C₁-C₁₂ heteroalkyl, wherein each of the alkyl or heteroalkyl is optionally substituted with one or more R^{5a}. In some embodiments, R⁵ is C₁-C₆ alkyl, C₁-C₆ haloalkyl, C₁-C₆ hydroxyalkyl, C₁-C₆ aminoalkyl, C₁-C₆ heteroalkyl, wherein each of the alkyl or heteroalkyl is optionally substituted with one or more R^{5a}. In some embodiments, R⁵ is C₁-C₁₂ alkyl, or C₁-C₁₂ heteroalkyl, wherein each of the alkyl or heteroalkyl is optionally substituted with one or more R^{5a}. In some embodiments, R⁵ is C₁-C₁₂ alkyl, wherein the alkyl is optionally substituted with one or more R^{5a}. In some embodiments, R⁵ is C₁-C₁₂ heteroalkyl, wherein the heteroalkyl is optionally substituted with one or more R^{5a}. In some embodiments, R⁵ is C₁-C₆ alkyl, wherein the alkyl is optionally substituted with one or more R^{5a}. In some embodiments, R⁵ is C₁-C₆ alkyl, wherein the alkyl is optionally substituted with one or more R^{5a} and R^{5a} is halogen, oxo, or —C(═O)O—C₁-₃ alkyl. In some embodiments, R⁵ is C₁-C₆ alkyl, wherein the alkyl is optionally substituted with one or more R^{5a} and R^{5a} is —C(═O)O—C₁-₃ alkyl. In some embodiments, R⁵ is C₁-C₆ alkyl, wherein the alkyl is optionally substituted with one or more R^{a} and R^{5a} is —C(═O)O—CH₃, or —C(═O)O—C(CH₃)₃. In some embodiments, R⁵ is C₁-C₆ heteroalkyl, wherein the heteroalkyl is optionally substituted with one or more R^{5a}. In some embodiments, R⁵ is C₁-C₆ heteroalkyl, wherein the heteroalkyl is optionally substituted with one or more R^{5a}, and R^{5a} is is halogen, oxo, or —C(═O)O—C₁-₃ alkyl. In some embodiments, R⁵ is C₁-C₆ heteroalkyl, wherein the heteroalkyl is optionally substituted with one or more R^{5a}, and R^{5a}—C(═O)O—C₁-₃ alkyl. In some embodiments, R⁵ is C₁-C₆ heteroalkyl, wherein the heteroalkyl is optionally substituted with one or more R^{5a}, and R^{5a}—C(═O)O—CH₃. In some embodiments, R⁵ is —(CH₂)₃—S—CH₃, wherein the heteroalkyl is optionally substituted with one or more R^{5a}. In some embodiments, R⁵ is

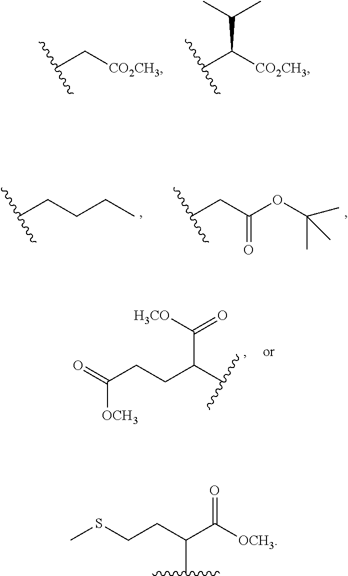

In some embodiments, R⁵ is

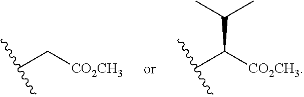

In some embodiments, R⁵ is -L²-cycloalkyl, -L⁵-heterocycloalkyl, -L⁵-aryl, or -L⁵-heteroaryl, wherein each of the cycloalkyl, heterocycloalkyl, aryl or heteroaryl is optionally substituted with one or more R^{5a}. In some embodiments, R⁵ is -L⁵-cycloalkyl, -L²-heterocycloalkyl, -L⁵-aryl, or -L⁵-heteroaryl, wherein each of the cycloalkyl, heterocycloalkyl, aryl or heteroaryl is optionally substituted with one or more substituents selected from halogen, oxo, —OH, C₁-C₆ alkyl, C₁-C₆ haloalkyl, and C₁-C₆ alkoxy. In some embodiments, R⁵ is -L⁵-cycloalkyl, -L⁵-heterocycloalkyl, -L⁵-aryl, or -L⁵-heteroaryl, wherein each of the cycloalkyl, heterocycloalkyl, aryl or heteroaryl is optionally substituted with one or more substituents selected from halogen, oxo, —OH, C₁-C₆ alkyl, C₁-C₆ haloalkyl, and C₁-C₆ alkoxy. In some embodiments, R⁵ is -L₅-cycloalkyl, -L⁵-heterocycloalkyl, -L⁵-phenyl, or -L⁵-indolyl, wherein each of the cycloalkyl, heterocycloalkyl, phenyl or indolyl is optionally substituted with one or more R^{5a}. In some embodiments, R⁵ is -L⁵-cycloalkyl, -L⁵-heterocycloalkyl, -L⁵-phenyl, or -L⁵-indolyl, wherein each of the cycloalkyl, heterocycloalkyl, phenyl or indolyl is optionally substituted with one or more R^{5a} and L⁵ is C₁-C₆ alkylene or C₁-C₆ heteroalkylene, each of which is optionally substituted with one or more R^{5a}. In some embodiments, R⁵ is -L⁵-cycloalkyl, -L⁵-heterocycloalkyl, -L⁵-phenyl, or -L⁵-indolyl, wherein each of the cycloalkyl, heterocycloalkyl, phenyl or indolyl is optionally substituted with one or more substituents selected from halogen, oxo, —OH, C₁-C₆ alkyl, C₁-C₆ haloalkyl, and C₁-C₆ alkoxy. In some embodiments, R⁵ is -L⁵-phenyl or -L⁵-indolyl each is optionally substituted with one or more R^{5a}. In some embodiments, R⁵ is -L⁵-phenyl or -L⁵-indolyl each optionally substituted with one or more R^{5a} and L⁵ is C₁-C₆ alkylene or C₁-C₆ heteroalkylene, each of which is optionally substituted with one or more R^{5a}. In some embodiments, R⁵ is -L⁵-phenyl or -L⁵-indolyl each optionally substituted with one or more substitutents selected from halogen, oxo, —OH, C₁-C₆ alkyl, C₁-C₆ haloalkyl, and C₁-C₆ alkoxy. In some embodiments, R⁵ is -L⁵-phenyl or -L⁵-indolyl each optionally substituted with one or more R^{5a}, wherein R^{5a} is halogen, oxo, —OH, or C₁-C₆ alkyl. In some embodiments, R⁵ is -L⁵-phenyl or -L⁵-indolyl each optionally substituted with one or more R, wherein R^{5a} is halogen, —OH, or oxo. In some embodiments, R⁵ is -L⁵-phenyl or -L⁵-indolyl, wherein the phenyl or indolyl are each unsubstituted. In some embodiments, R⁵ is -L⁵-phenyl or -L⁵-indolyl, wherein the phenyl or indolyl are each unsubstituted and L⁵ is C₁-C₆ alkylene or C₁-C₆ heteroalky lene, each of which is optionally substituted with one or more R^{5a}. In some embodiments, R⁵ is -L⁵-phenyl or -L⁵-indolyl, wherein the phenyl or indolyl are each unsubstituted and L⁵ is C₁-C₆ alkylene or C₁-C₆ heteroalkylene, each of which is optionally substituted with one or more R^{5a}, and R^{5a} is —C(═O)O—CH₃. In some embodiments, R⁵ is -L⁵-phenyl or -L⁵-indolyl, wherein the phenyl or indolyl are each unsubstituted and L⁵ is —CH₂—, —CH₂CH₂—, or —CH₂CH₂—O—CH₂—, each of which is optionally substituted with one or more R^{5a}, and R^{5a} is —C(═O)O—CH₃. In some embodiments, R⁵ is

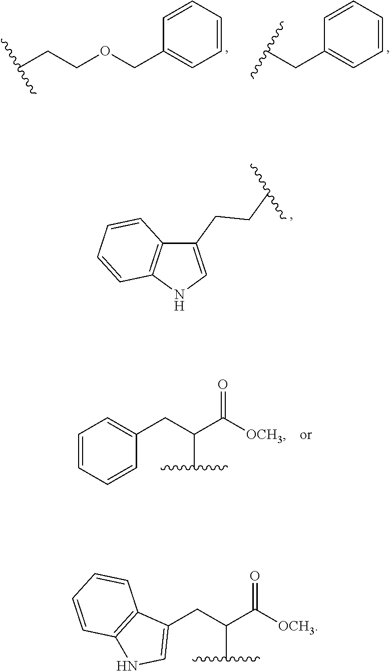

L⁵ is any suitable linker known by one of skill in the art. In some embodiments, L⁵ is absent, C₁-C₆ alkylene, C₁-C₆ heteroalkylene, C₂-C₆ alkenylene, or C₂-C₂₄ alkynylene, each of which is optionally substituted with one or more R^{5a}. In some embodiments, L⁵ is absent, C₁-C₆ alkylene, C₁-C₆ heteroalkylene, or C₂-C₆ alkenylene, each of which is optionally substituted with one or more R^{5a}. In some embodiments. L⁵ is C₁-C₆ alkylene or C₁-C₆ heteroalkylene, each of which is optionally substituted with one or more R^{5a}. In some embodiments, L⁵ is C₁-C₆ alkylene or C₁-C₆ heteroalkylene. each of which is optionally substituted with one or more R^{5a}, wherein R^{5a} is —C(═O)OR^{a}. In some embodiments, L⁵ is C₁-C₆ alkylene optionally substituted with one or more R^{5a}. In some embodiments, L⁵is C₁-C₆ alkylene optionally substituted with one or more R^{a}, wherein R^{5a} is —C(═O)OR^{a}. In some embodiments, L⁵ is C₁-C₆ alkylene optionally substituted with one or more R^{5a}, wherein R^{5a} is —C(═O)O—CH₃. In some embodiments, L⁵ is C₁-C₆ alkylene. In some embodiments, L⁵ is C₁-C₃ alkylene. In some embodiments, L⁵ is L⁵is C₁-C₆ heteroalkylene optionally substituted with one or more R^{5a}. In some embodiments, L⁵ is C₁-C₆ heteroalkylene optionally substituted with one or more R^{5a}, wherein R^{5a} is —C(═O)OR^{a}. In some embodiments, L⁵is C₁-C₆ heteroalkylene optionally substituted with one or more R^{5a}, wherein R^{5a} is —C(═O)O—CH₃. In some embodiments, L⁵ is L⁵ is C₁-C₆ heteroalkylene. In some embodiments, L⁵ is L⁵ is C₁-C₃ heteroalkylene.

R^{5a} can be any suitable functional group known by one of skill in the art. In some embodiments, each R^{5a} is independently halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R. In some embodiments, each R^{5a} is independently halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl. In some embodiments, R^{5a} is halogen, —CN, —NO₂, —OH, —OR^{a}, oxo, —OC(═O)R^{a}, —OC(═O)OR^{b}, —SH, —SR^{a}, —S(═O)R^{a}, —S(═O)₂R^{a}, —NR^{c}R^{d}, —C(═O)R^{a}, —C(═O)OR^{a}, C₁-C₆ alkyl, C₁-C₆ haloalkyl, C₁-C₆ hydroxyalkyl, C₁-C₆ aminoalkyl, C₁-C₆ heteroalkyl, cycloalkyl, or heterocycloalkyl, wherein each of the alkyl, heteroalkyl, cycloalkyl, or heterocycloalkyl is optionally substituted with one or more R. In some embodiments, In some embodiments, R^{5a} is halogen, —OR^{a}, oxo, —C(═O)OR^{a}, C₁-C₆ alkyl, or C₁-C₆ heteroalkyl, wherein each of the alkyl or heteroalkyl is optionally substituted with one or more R. In some embodiments, R^{5a} is halogen, oxo, —C(═O)O—C₁-₆ alkyl, C₁-C₆ alkyl, C₁-C₆ haloalkyl, C₁-C₆ alkoxy, C₃-C₆ cycloalkyl, or C₂-C₅ heterocycloalkyl. In some embodiments, R^{5a} is halogen, oxo, —C(═O)O—C₁-₃ alkyl, C₁-C₆ alkyl, C₁-C₆ haloalkyl, C₁-C₆ alkoxy, C₃-C₆ cycloalkyl, or C₂-C₅ heterocycloalkyl. In some embodiments, R^{5a} is oxo, —C(═O)O—C₁-₃ alkyl, or C₂-C₅ heterocycloalkyl. In some embodiments, R^{5a} is, oxo, —C(═O)O—CH₃, —C(═O)O—C(CH₃)₃ or C₅ heterocycloalkyl. In some embodiments, R^{5a} is —C(═O)O—CH₃, —C(═O)O—C(CH₃)₃ or piperadinyl.

In some embodiments, R⁴ and R⁵ are taken together with the nitrogen to which they are attached to form a heterocycloalkyl or heteroaryl. In some embodiments, R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a}. In some embodiments, R⁴ and R⁵ are taken together with the nitrogen to which they are attached to form a heterocycloalkyl or heteroaryl. In some embodiments, R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a}, wherein R^{5a} is halogen, oxo, —C(═O)O—C₁-₃ alkyl, C₁-C₆ alkyl, C₁-C₆ haloalkyl, C₁-C₆ alkoxy, C₃-C₆ cycloalkyl, or C₂-C₅ heterocycloalkyl. In some embodiments, R⁴ and R⁵ are taken together with the nitrogen to which they are attached to form a 5 to 8 membered heterocycloalkyl or heteroaryl, wherein each of the heterocycloalkyl or heteroaryl is optionally substituted with one or more R^{5a}. In some embodiments, R⁴ and R⁵ are taken together with the nitrogen to which they are attached to form a 5 to 8 membered heterocycloalkyl or heteroaryl, wherein each of the heterocycloalkyl or heteroaryl is optionally substituted with one or more R^{5a}, wherein R^{5a} is halogen, oxo, —C(═O)O—C₁-₃ alkyl, C₁-C₆ alkyl, C₁-C₆ haloalkyl, C₁-C₆ alkoxy, C₃-C₆ cycloalkyl, or C₂-C₅ heterocycloalkyl. In some embodiments, R⁴ and R⁵ are taken together with the nitrogen to which they are attached to form a 5 or 6 membered heterocycloalkyl or heteroaryl, each of which is optionally substituted. In some embodiments, R⁴ and R⁵ are taken together with the nitrogen to which they are attached to form a 5 or 6 membered heterocycloalkyl or heteroaryl, each of which is optionally substituted and contains 1 or 2 nitrogen and 0-1 oxygen. In some embodiments, R⁴ and R⁵ are taken together with the nitrogen to which they are attached to form a 5 to 6 membered heterocycloalkyl. In some embodiments, R⁴ and R⁵ are taken together with the nitrogen to which they are attached to form a 5 to 6 membered heterocycloalkyl each of which is optionally substituted and contains 1 or 2 nitrogen and 0-1 oxygen. R⁴ and R⁵ are taken together with the nitrogen to which they are attached to form a 5 to 6 membered heterocycloalkyl each of which is optionally substituted with one or more R^{5a}. In some embodiments, R⁴ and R⁵ are taken together with the nitrogen to which they are attached to form a 5 to 6 membered heterocycloalkyl each of which is optionally substituted with one or more R^{5a}, wherein R^{5a} is halogen, oxo, —C(═O)O—C₁-₃ alkyl, C₁-C₆ alkyl, C₁-C₆ haloalkyl, C₁-C₆ alkoxy, C₃-C₆ cycloalkyl, or C₂-C₅ heterocycloalkyl. In some embodiments, R⁴ and R⁵ are taken together with the nitrogen to which they are attached to form a 5 to 6 membered heterocycloalkyl each of which is optionally substituted with one or more R^{5a}, wherein R^{5a} is —C(═O)O—C₁-₃ alkyl or C₂-C₅ heterocycloalkyl. In some embodiments, R⁴ and R⁵ are taken together with the nitrogen to which they are attached to form a pyrrolidinyl, morpholinyl, or piperidinyl, each of which is optionally substituted with one or more R^{5a}. In some embodiments, R⁴ and R⁵ are taken together with the nitrogen to which they are attached to form a pyrrolidinyl, morpholinyl, or piperidinyl, each of which is optionally substituted with one or more R^{5a}, wherein R^{5a} is —C(═O)O—C₁-₃ alkyl or C₂-C₅ heterocycloalkyl. In some embodiments, R⁴ and R⁵ are taken together with the nitrogen to which they are attached to form a pyrrolidinyl, morpholinyl, or piperidinyl, each of which is optionally substituted with one or more R^{5a}, wherein R^{5a} is —C(═O)O—CH₃ or piperidinyl. In some embodiments, R⁴ and R⁵ are taken together with the nitrogen to which they are attached to form a morpholinyl or piperidinyl, each of which is optionally substituted with one or more R^{5a}, wherein R^{5a} is piperidinyl. In some embodiments, the structure

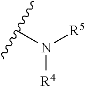

is

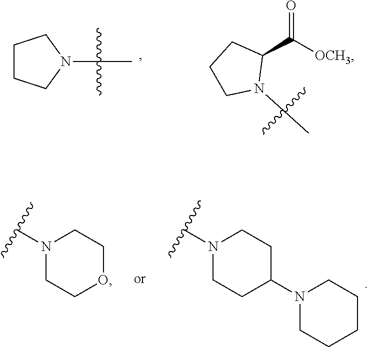

In some embodiments, the structure

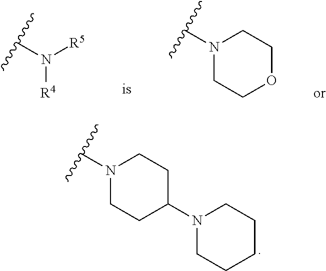

In some embodiments, the structure

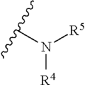

is

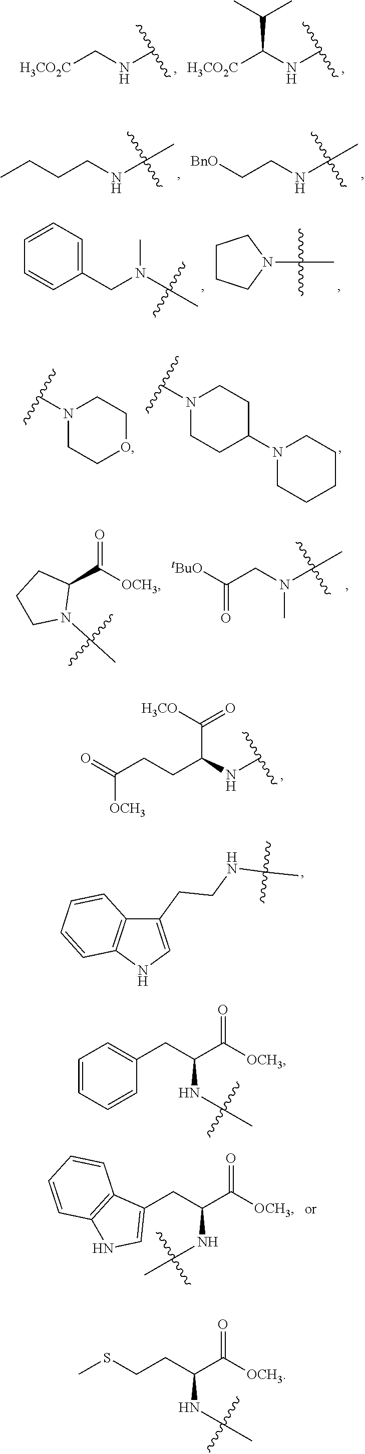

In some embodiments, the structure

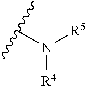

is

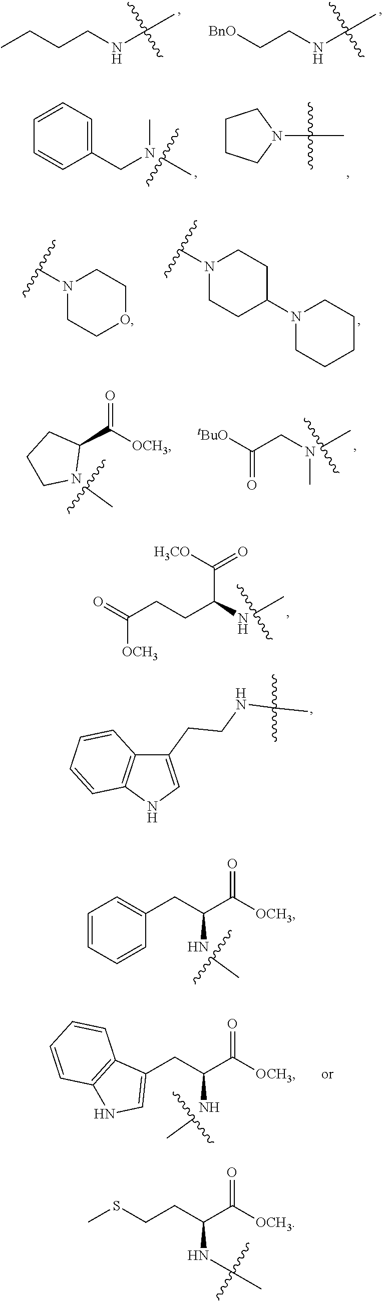

In some embodiments, the structure

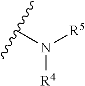

is

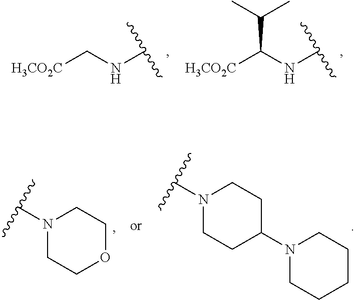

In some embodiments, Q is: (S)-1-carboxyethylamino, (S)-1-carboxy-4-guanidinobutylamino, (S)-3-amino-1-carboxy-3-oxopropylamino, (S)-1,2-dicarboxyethylamino, (S)-1-carboxy-2-mercaptoethylamino, (S)-4-amino-1-carboxy-4-oxobutylamino, (S)-3-carboxy-1-carboxylatepropylamino, (S)-1-carboxy-2-(1H-imidazol-4-yl)ethylamino, (1S,2S)-1-carboxy-2-methylbutylamino, (S)-1-carboxy-3-methylbutylamino, (S)-5-amino-1-carboxypentylamino, (S)-1-carboxy-3-(methylthio)propylamino, (S)-1-carboxy-2-phenylethylamino, (S)-2-carboxypyrrolidin-1-yl, (S)-1-carboxy-2-hydroxyethylamino, (1S,2R)-1-carboxy-2-hydroxypropylamino, (S)-1-carboxy-2-(1H-indol-3-yl)ethylamino, (S)-1-carboxy-2-(4-hydroxyphenyl)ethylamino or (S)-1-carboxy-2-methylpropylamino.

In some embodiments, the compound, or a pharmaceutically acceptable salt or solvate thereof, has a structure of Formula (IV)

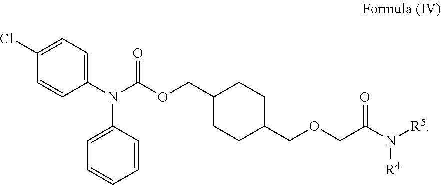

In some embodiments, the compound, or a pharmaceutically acceptable salt or solvate thereof, has a structure of Formula (IVa)

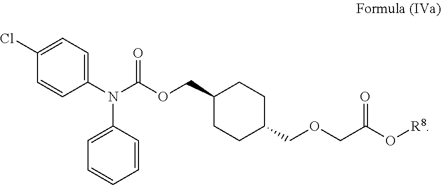

R⁸ can be any suitable functional group known by one of skill in the art. In some embodiments, R⁸ is —CH₂CH₂CH₃, —CH(CH₃)₂, —CH₂CH₂CH₂CH₃, —CH₂CH(CH₃)₂, C₅-C₂₄ alkyl, C₁-C₂₄ haloalkyl, C₁-C₂₄ heteroalkyl, C₂-C₂₄ alkenyl, C₂-C₂₄ alkynyl, -L³-—-P(═O)(OH)₂, -L³-cycloalkyl, -L³-heterocycloalkyl, or -L³-aryl, wherein each of the alkyl, heteroalkyl, alkenyl, alkynyl, cycloalkyl, heterocycloalkyl, or aryl is optionally substituted with one or more R^{8a}. In some embodiments, R⁸ is —CH₂CH₂CH₃, —CH(CH₃)₂, —CH₂CH₂CH₂CH₃, —CH₂CH(CH₃)₂, C₅-C₂₄ alkyl, C₁-C₂₄ haloalkyl, C₁-C₂₄ heteroalkyl, C₂-C₂₄ alkenyl, C₂-C₂₄ alkynyl, -L³-O—P(═O)(OH)₂, -L³-cycloalkyl, -L³-heterocycloalkyl, or -L³-aryl. In some embodiments, R⁸ is C₅-C₂₄ alkyl, C₂-C₂₄ alkenyl, C₂-C₂₄ alkynyl, -L³-O—P(═O)(OH)₂, or -L³-aryl, wherein each of the alkyl, alkenyl, alkynyl, or aryl is optionally substituted with one or more R^{8a}. In some embodiments, R⁸ is C₅-C₂₄ alkyl, C₂-C₂₄ alkenyl, C₂-C₂₄ alkynyl, -L³-O—P(═O)(OH)₂, or -L³-aryl. In some embodiments, R⁸ is C₃-C₁₂ alkyl, C²-C¹² alkenyl, C²-C¹²alkynyl, L³-(5 or 6-membered cycloalkyl), -L³-(5 or 6-membered heterocycloalkyl), -L3-phenyl, -L³-naphthyl, or -L³-heteroaryl, wherein each of the alkyl, heteroalkyl, alkenyl, alkynyl, cycloalkyl, heterocycloalkyl, phenyl, naphthyl or heteroaryl is optionally substituted with one or more R^{8a}.

In some embodiments, R⁸ is —CH₂CH₂CH₃, —CH(CH₃)₂, —CH₂CH₂CH₂CH₃, —CH₂CH(CH₃)₂, C₅-C₁₄ alkyl, C₁-C₁₂ heteroalkyl, C₂-C₁₂ alkenyl, or C₂-C₁₂ alkynyl, wherein each of the alkyl, heteroalkyl, alkenyl, or alkynyl is optionally substituted with one or more R^{8a}. In some embodiments, R⁸ is —CH₂CH₂CH₃, —CH(CH₃)₂, —CH₂CH₂CH₂CH₃, —CH₂CH(CH₃)₂, C₅-C₁₂ alkyl, C₁-C₁₂ heteroalkyl, C₂-C₁₂ alkenyl, or C₂-C₁₂alkynyl, wherein each of the alkyl, heteroalkyl, alkenyl, or alkynyl is optionally substituted with one or more R^{8a}. In some embodiments, R⁸ is —CH₂CH₂CH₃, —CH(CH₃)₂, —CH₂CH₂CH₂CH₃, —CH₂CH(CH₃)₂, C₅-C₁₂ alkyl, C₁-C₁₂ heteroalkyl, C₂-C₁₂ alkenyl, or C₂-C₁₂alkynyl. In some embodiments, R⁸ —CH₂CH₂CH₂CH₃, C₅-C₁₂ alkyl, C₁-C₆ heteroalkyl, C₂-C₁₂ alkenyl, or C₂-C₁₂alkynyl, wherein each of the alkyl, heteroalkyl, alkenyl, or alkynyl is optionally substituted with one or more R^{8a}, and R^{8a} is oxo. In some embodiments, R⁸ —CH₂CH₂CH₂CH₃, C₅-C₁₂ alkyl, C₁-C₆ heteroalkyl each optionally substituted with one or more R^{8a}, and R^{8a} is oxo.

In some embodiments, R⁸ is

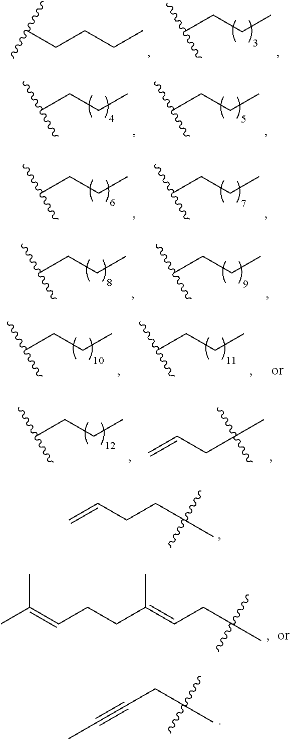

In some embodiments, R⁸ is

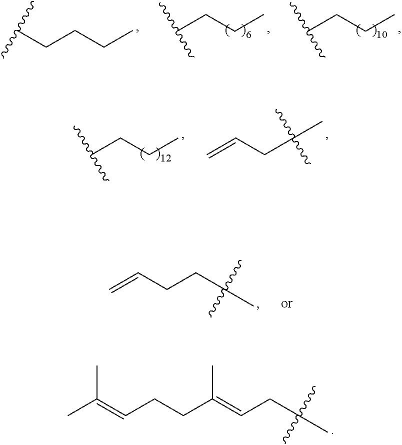

In some embodiments, R⁸ is

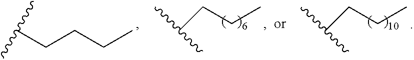

In some embodiments, R⁸ is

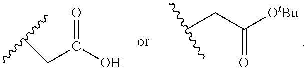 [SMILES: *CC(=O)O ; *CC(=O)OC(C)(C)C]

In some embodiments, R³ is

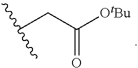 [SMILES: *CC(=O)OC(C)(C)C]

In some embodiments, R⁸ is -L³-(5 or 6-membered cycloalkyl), -L³-(5 or 6-membered heterocycloalkyl), -L³-phenyl, -L³-naphthyl, or -L³-heteroaryl, each of the cycloalkyl, heterocycloalkyl, phenyl, naphthyl or heteroaryl is optionally substituted with one or more R^{8a}. In some embodiments, R⁸ is -L³-(5 or 6-membered cycloalkyl), -L³-(5 or 6-membered heterocycloalkyl), -L³-phenyl, -L³-naphthyl, or -L³-heteroaryl, each of the cycloalkyl, heterocycloalkyl, phenyl, naphthyl or heteroaryl is optionally substituted with one or more R^{8a}; L3 is absent, C₂-C₆ alkenylene, C₁-C₆ alkylene or C₁-C₆ heteroalkylene, each of which is optionally substituted. In some embodiments, R⁸ is -L³-(5 or 6-membered cycloalkyl), -L³-phenyl, -L³-naphthyl, or -L³-heteroaryl, each of the cycloalkyl, phenyl, naphthyl or heteroaryl is optionally substituted with one or more R^{8a}. In some embodiments, R⁸ -L³-(5 or 6-membered cycloalkyl), -L³-(5 or 6-membered heterocycloalkyl), -L²-phenyl, -L³-naphthyl, or -L³-heteroaryl, each of the cycloalkyl, phenyl, naphthyl or heteroaryl is optionally substituted with one or more R^{8a}, wherein R^{8a} is halogen, oxo, C₁-C₆ alkyl, or C₁-C₆ haloalkyl, and L³ is absent C₁-C₆ alkylene, C₁-C₃ heteroalkylene, or C₂-C₃ alkenylene. In some embodiments, R⁸ -L³-cyclohexyl, -L³-(1,3-dioxolyl), -L³-phenyl, or -L³-naphthyl, each optionally substituted with one or more R^{8a}. In some embodiments, R⁸ -L²-cyclohexyl, -L³-(1,3-dioxolyl), -L²-phenyl, or -L³-naphthyl, each optionally substituted with one or more R^{8a}, wherein R^{8a} is halogen oxo, C₁-C₆ alkyl, or C₁-C₆ haloalkyl, and L³ is absent C₁-C₆ alkylene, C₁-C₃ heteroalkylene, or C₂-C₃ alkenylene. In some embodiments, R⁸ -L³-cyclohexyl, -L³-(1,3-dioxolyl), -L³-phenyl, or -L³-naphthyl, each optionally substituted with one or more R^{8a}, wherein R^{8a} is fluoro, oxo, —CH₃, or —CF₃. In some embodiments, R⁸ -L³-cyclohexyl, -L³-(1,3-dioxolyl), -L³-phenyl, or -L³-naphthyl, each optionally substituted with one or more R^{8a}, wherein R^{8a} is fluoro oxo, —CH₃, or —CF₃, and wherein L³ is absent C₁-C₆ alkylene, C₁-C₃ heteroalkylene, or C₂-C₃alkenylene. In some embodiments, R⁸ -L³-cyclohexyl, -L³-(1,3-dioxolyl), -L³-phenyl, or -L³-naphthyl, each optionally substituted with one or more R^{8a}, wherein R^{8a} is fluoro. oxo, —CH₃, or —CF₃, and wherein L³ is absent, —CH₂CH₂—, —CH₂CH₂—O—, —CH₂—or —CH₂CH═CH—.

In some embodiments, R⁸ is

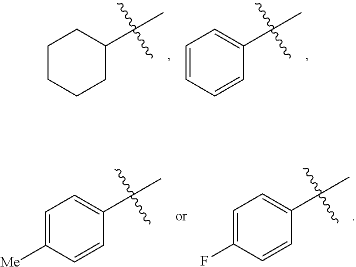 [SMILES: *C1CCCCC1 ; *c1ccccc1 ; *c1ccc(C)cc1 ; *c1ccc(F)cc1]

In some embodiments, R⁸ is

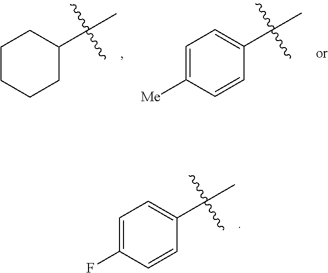 [SMILES: *C1CCCCC1 ; *c1ccc(C)cc1 ; *c1ccc(F)cc1]

In some embodiments, R⁸ is

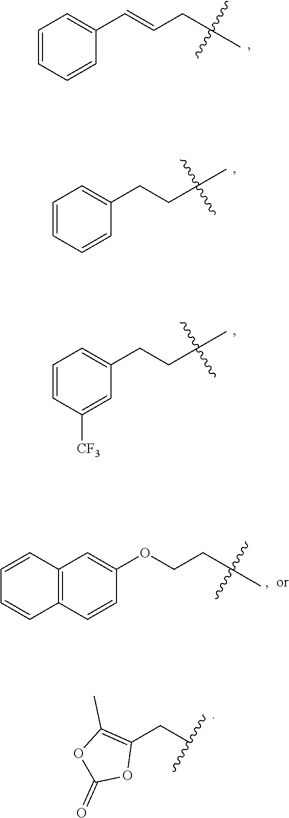

In some embodiments, R⁸ is

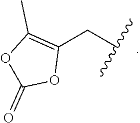 [SMILES: *Cc1oc(=O)oc1C]

In some embodiments, R⁸ is -L³-O—P(═O)(OH)₂. In some embodiments, R⁸ is

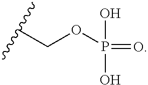 [SMILES: *COP(=O)(O)O]

L³ is any suitable linker known by one of skill in the art. In some embodiments, L³ is absent, C₁-C₆ alkylene, C₁-C₆ heteroalkylene, C₂-C₆ alkenylene, or C₂-C₂₄ alkynylene, each of which is optionally substituted with one or more R^{8a}. In some embodiments, L³ is absent, C₁-C₆ alkylene, C₁-C₆ heteroalkylene, or C₂-C₆ alkenylene. In some embodiments, L³ is absent C₁-C₆ alkylene, C₁-C₃ heteroalkylene, or C₂-C₃ alkenylene. In some embodiments, L³ is absent, —CH₂CH₂—, —CH₂CH₂—O—, —CH₂— or —CH₂CH═CH—. In some embodiments, L³ is absent.

R^{8a} can be any suitable functional group known by one of skill in the art. In some embodiments, R^{8a} is halogen, —CN, —NO₂, —OH, —OR^{a}, oxo, —OC(═O)R^{a}, —SH, —SR^{a}, —S(═O)R^{a}, —S(═O)₂R^{a}, —S(═O)₂OR^{b}, —S(—O)₂NR^{c}R^{d}, —NR^{c}R^{d}, —NR^{b}C(═O)₂R^{a}, —NR^{b}S(═O)₂R^{a}, —C(═O)R^{a}, —C(═O)OR^{b}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R. In some embodiments, R^{8a} is halogen, —CN, —NO₂, —OH, —OR^{a}, oxo, —OC(═O)R^{a}, —OC(═O)OR^{b}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, C₁-C₆ alkyl, C₁-C₆ haloalkyl, C₁-C₆ heteroalkyl, cycloalkyl, heterocycloalkyl, aryl, or heteroaryl, wherein each of the alkyl, heteroalkyl, cycloalkyl, heterocycloalkyl, aryl, or heteroaryl is optionally substituted with one or more R. In some embodiments, R^{8a} is halogen, —CN, —NO₂, —OH, —OR^{a}, oxo, —OC(═O)R^{a}, —OC(═O)OR^{b}, —SH, —SRₐ, —S(═O)R^{a}, —S(═O)₂R^{a}, —S(═O)₂NR^{c}R^{d}, —NR^{c}R^{d}, —C(═O)R^{a}, —C(═O)OR^{a}, C₁-C₆ alkyl, C₁-C₆ haloalkyl, C₁-C₆ heteroalkyl, cycloalkyl, heterocycloalkyl, aryl, or heteroaryl. In some embodiments, R^{8a} is halogen, —OR^{a}, oxo, C₁-C₆ alkyl, or C₁-C₆ haloalkyl, wherein each of the alkyl or haloalkyl is optionally substituted with one or more R. In some embodiments, R^{8a} is halogen, —OR^{a}, oxo, C₁-C₆ alkyl, or C₁-C₆ haloalkyl, wherein each of the alkyl or haloalkyl is optionally substituted with one or more R. In some embodiments, R^{8a} is halogen, oxo, C₁-C₆ alkyl, C₁-C₆ haloalkyl, or C₁-C₆ alkoxy. In some embodiments, R^{8a} is fluoro, oxo, —CH₃, —CF₃, or C₁-C₃ alkoxy. In some embodiments, R^{8a} is fluoro, oxo, —CH₃, or —CF₃

R can be any suitable functional group known by one of skill in the art. In some embodiments, R is independently halogen, —CN, —OH, oxo, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆heteroalkyl. In some embodiments, R is independently halogen, —CN, —OH, oxo, —OC₁-C₆alkyl, —NH₂, —C(═O)OH, or —C(═O)OC₁-C₆alkyl. In some embodiments, R is independently halogen, —OH, oxo, —OC₁-C₆alkyl, —C(═O)OH, or —C(—O)OC₁-C₆alkyl. In some embodiments, R is independently oxo.

R can be any suitable functional group known by on eof skill in the art. In some embodiments, each R is independently halogen, —CN, —OH, oxo, —OC₁-C₆alkyl, —S(═O)C₁-C₆alkyl, —S(═O)₂C₁-C₆alkyl, —S(═O)₂NH₂, —S(—O)₂NHC₁-C₆alkyl, —S(═O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆heteroalkyl. In some embodiments, each R is independently halogen, —CN, —OH, oxo, or -OC₁-C₆alkyl. In some embodiments, each R is independently halogen, —CN, —OH, or oxo. In some embodiments, each R is independently oxo.

In some embodiments of a compound disclosed herein, one or more of R, R¹, R², R³, R⁴, R⁵, R^{5a}, R⁶, R⁷, R^{7a}, R⁸, R^{8a}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R⁴, R⁵, R^{5a}, R⁶, R⁷, R^{7a}, R⁸, R^{8a}, R^{a}, R^{b}, R^{c}, and R^{d}.

In some embodiments of a compound disclosed herein, the abundance of deuterium in each of R, R¹, R², R³, R⁴, R⁵, R^{5a}, R⁶, R⁷, R^{7a}, R⁸, R^{8a}, R^{a}, R^{b}, R^{c}, and/or R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more ¹H of cycloalkyl, heterocycloalkyl, aryl, or heteroaryl are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt, solvate, or stereoisomer thereof, is one of the compounds in Table 1.

TABLE 1
Ralinepag Prodrugs

| | | | HPLC |
|---|---|---|---|
| Cmpd No. | Compound Structure | MW | Purity (%) |
| 2a | 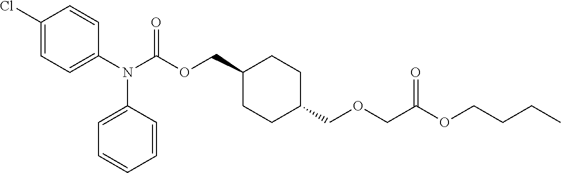 [SMILES: CCCCOC(=O)COC[C@H]1CC[C@H](COC(=O)N(c2ccccc2)c2ccc(Cl)cc2)CC1] | 488.02 | 99.14 |
| 2b | 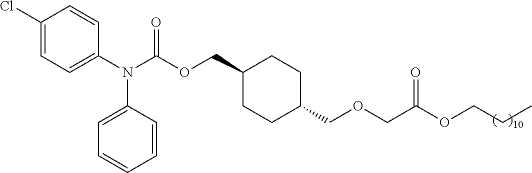 | 600.24 | 96.68 |
| 2c | 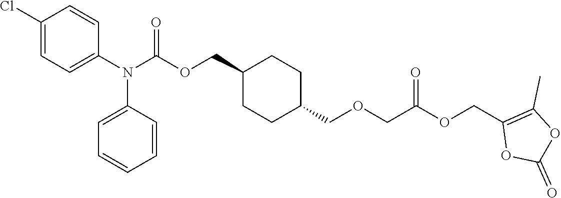 [SMILES: Cc1oc(=O)oc1COC(=O)COC[C@H]1CC[C@H](COC(=O)N(c2ccccc2)c2ccc(Cl)cc2)CC1] | 544.00 | 96.29 |
| 4a | 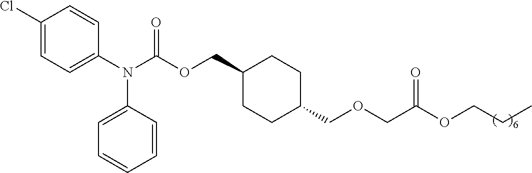 | 544.13 | 99.16 |
| 4b | 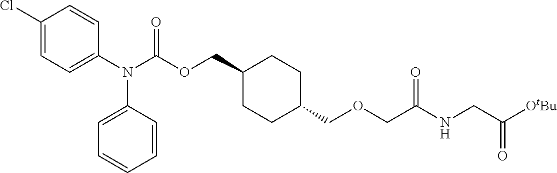 [SMILES: CC(C)(C)OC(=O)CNC(=O)COC[C@H]1CC[C@H](COC(=O)N(c2ccccc2)c2ccc(Cl)cc2)CC1] | 546.06 | 99.17 |
| 6 | 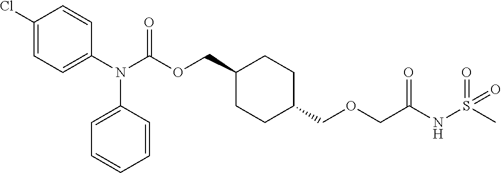 [SMILES: CS(=O)(=O)NC(=O)COC[C@H]1CC[C@H](COC(=O)N(c2ccccc2)c2ccc(Cl)cc2)CC1] | 509.01 | 98.43 |
| 8a | 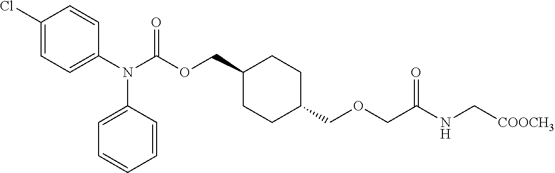 [SMILES: COC(=O)CNC(=O)COC[C@H]1CC[C@H](COC(=O)N(c2ccccc2)c2ccc(Cl)cc2)CC1] | 502.99 | 99.51 |
| 8b | 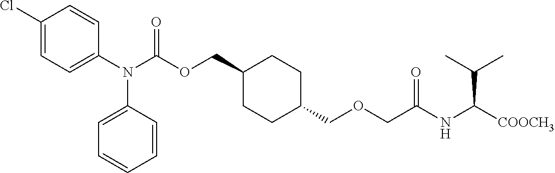 [SMILES: COC(=O)[C@@H](NC(=O)COC[C@H]1CC[C@H](COC(=O)N(c2ccccc2)c2ccc(Cl)cc2)CC1)C(C)C] | 545.07 | 98.20 |
| 8c | 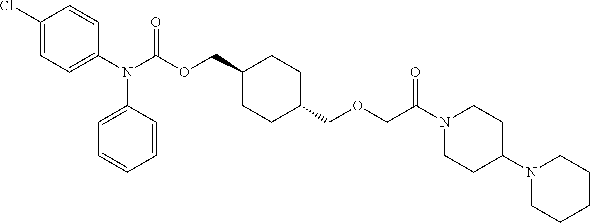 [SMILES: O=C(COC[C@H]1CC[C@H](COC(=O)N(c2ccccc2)c2ccc(Cl)cc2)CC1)N1CCC(N2CCCCC2)CC1] | 582.18 | 98.27 |
| 9 | 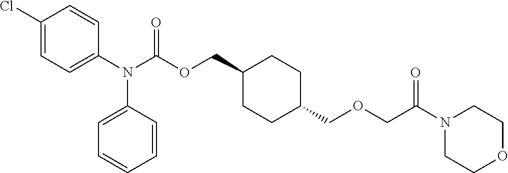 [SMILES: O=C(COC[C@H]1CC[C@H](COC(=O)N(c2ccccc2)c2ccc(Cl)cc2)CC1)N1CCOCC1] | 501.02 | 99.62 |

In some embodiments, the compound disclosed herem, or a pharmaceutically acceptable salt, solvate, or stereoisomer thercof, is one of the compounds in Table 2.

TABLE 2
Other Prodrugs Moieties

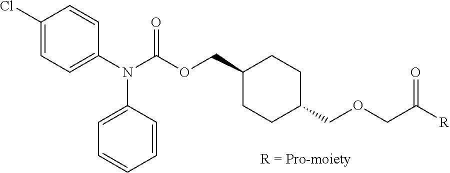

R

| Sulfonamide Pro-moiety | Amide Pro-moiety | Ester Pro-moiety |
|---|---|---|
| 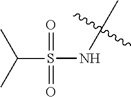 [SMILES: *NS(=O)(=O)C(C)C] | 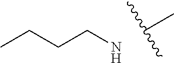 | 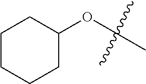 [SMILES: *OC1CCCCC1] |
| 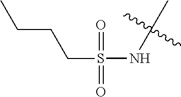 [SMILES: *NS(=O)(=O)CCCC] | 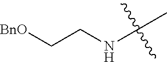 [SMILES: *NCCOCc1ccccc1] | 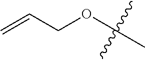 [SMILES: *OCC=C] |
| 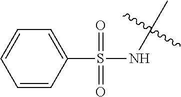 [SMILES: *NS(=O)(=O)c1ccccc1] | 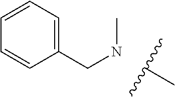 | 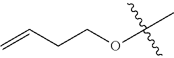 [SMILES: *OCCC=C] |
| 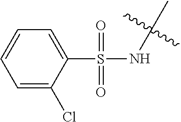 [SMILES: *NS(=O)(=O)c1ccccc1Cl] | 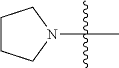 [SMILES: *N1CCCC1] | 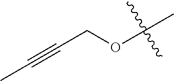 [SMILES: *OCC#CC] |
| 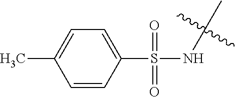 [SMILES: *NS(=O)(=O)c1ccc(C)cc1] | 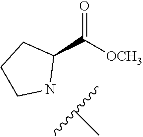 | 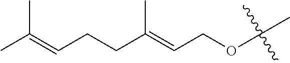 [SMILES: *OC/C=C(\C)CCC=C(C)C] |
| 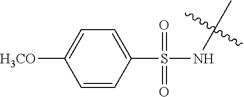 [SMILES: *NS(=O)(=O)c1ccc(OC)cc1] | 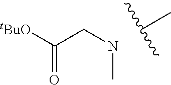 | 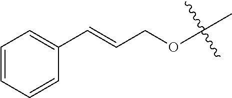 [SMILES: *OC/C=C/c1ccccc1] |
| 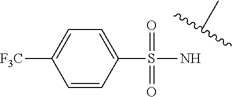 | 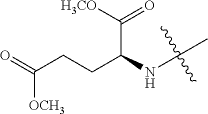 [SMILES: *N[C@@H](CCC(=O)OC)C(=O)OC] | 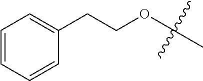 [SMILES: *OCCc1ccccc1] |
| 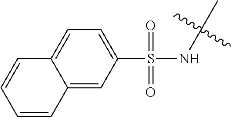 [SMILES: *NS(=O)(=O)c1ccc2ccccc2c1] | 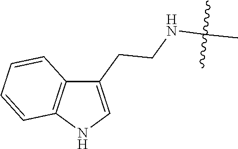 [SMILES: *NCCc1c[nH]c2ccccc12] | 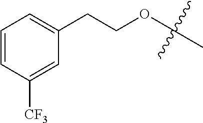 [SMILES: *OCCc1cccc(C(F)(F)F)c1] |
| 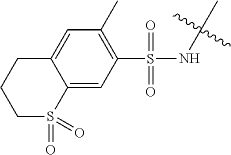 [SMILES: *NS(=O)(=O)c1cc2c(cc1C)CCCS2(=O)=O] | 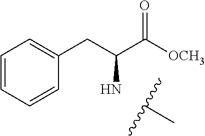 | 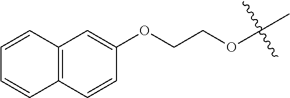 [SMILES: *OCCOc1ccc2ccccc2c1] |
| 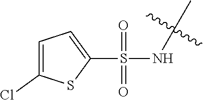 [SMILES: *NS(=O)(=O)c1ccc(Cl)s1] | 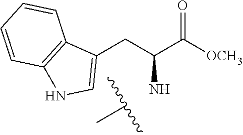 | 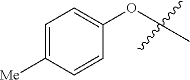 [SMILES: *Oc1ccc(C)cc1] |
| 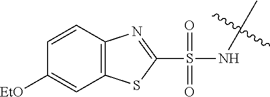 [SMILES: *NS(=O)(=O)c1nc2ccc(OCC)cc2s1] | 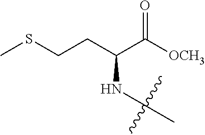 [SMILES: *N[C@@H](CCSC)C(=O)OC] | 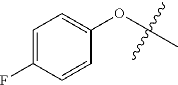 [SMILES: *Oc1ccc(F)cc1] |

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomerie compounds, separating the diastereomers and recovering the optically pure enantiomers. In some embodiments, dissociable complexes are preferred. In some embodiments, the diastercomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H(D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their case of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more ¹H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acry late, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxy butyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-cthanedisulfonic acid, 2-hydroxyethanesulfonic acid, benzenesulfonic acid, 2-naphthalenesulfonic acid, 4-methylbicyclo-[2,2,2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethy lamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quatemization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tantomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Pulmonary Arterial Hypertension (PAH), PGI₂, and IP Receptor Agonists

Pulmonary hypertension (PH) is a rare, progressive disease characterized by elevated pulmonary vascular resistance (PVR) that can lead to right ventricular enlargement, hypertrophy, failure and ultimately death. There are five different groups of PH based on different causes according to the current World Health Organization (WHO) classification. These are referred to as PH WHO Groups. Group I is pulmonary arterial hypertension (PAH), which is characterized by a thickening and stiffening of the pulmonary vasculature. Although management of PAH has improved significantly in the past 15 years, the mortality rate is still unacceptably high, with a median life expectancy of 7 years after diagnosis. WHO Group 2 includes PH due to left heart disease. In these patients, problems with the heart, rather than the pulmonary vasculature, are primarily responsible for the disease state. WHO Group 3 includes PH due to chronic lung disease and/or hypoxia (low oxygen levels). Group 3 includes pulmonary hypertension associated with interstitial lung disease (PH-ILD) and PH associated with pulmonary fibrosis. WHO Group 4 is called chronic thromboembolic pulmonary hypertension (CTEPH). WHO Group 5 is where PH is secondary to other diseases in ways that are not well understood. Treatment depends on the form of PH. For example. PAH is frequently treated with prostacyclins. Regardless of classification, PH is a serious and often fatal disease.

Severity of PH, including PAH, is graded by 4 functional classes according to a system originally developed for heart failure by the New York Heart Association (NYHA) and then modified by the WHO for patients with PAH. Patients are usually asymptomatic in the earliest stages of the disease (ie, functional class J), but as the disease progresses, their symptoms, which include exertional dyspnea, fatigue, peripheral edema, and syncope, can be indistinguishable from other cardiorespiratory diseases. Many patients are not diagnosed until they have developed symptoms of WHO/NYHA functional class II or III.

Pulmonary arterial hypertension (PAH) has a multifactorial pathobiology. Vasoconstriction, remodeling of the pulmonary vessel wall, and thrombosis contribute to increased pulmonary vascular resistance in PAH (Humbert et al., J. Am. Coll. Cardiol., 2004, 43:13 S-24S.)

The compounds disclosed herein can be useful in the treatment of pulmonary arterial hypertension (PAH) and symptoms thereof. PAH shall be understood to encompass all forms of pulmonary arterial hypertension described in the 2003 World Health Organization (WHO) clinical classification of pulmonary arterial hypertension. Those forms include idiopathic PAH (IPAH); familial PAH (FPAH); PAH associated with other conditions (APAH), such as PAH associated with collagen vascular disease, PAH associated with congenital systemic-to-pulmonary shunts, PAH associated with portal hypertension. PAH associated with HIV infection, PAH associated with drugs or toxins, or PAH associated with Other; and PAH associated with significant venous or capillary involvement.

Idiopathic PAH refers to PAH of undetermined cause.

Familial PAH refers to PAH for which hereditary transmission is suspected or documented.

PAH associated with collagen vascular disease shall be understood to encompass PAH associated with scleroderma, PAH associated with CREST (calcinosis cutis, Raynaud's phenomenon, esophageal dysfunction, sclerodactyl), and telangiectasias) syndrome, PAH associated with systemic lupus erythematosus (SLE), PAH associated with rheumatoid arthritis, PAH associated with Takayasu's arteritis, PAH associated with polymyositis, and PAH associated with dermatomyositis.

PAH associated with congenital systemic-to-pulmonary shunts shall be understood to encompass PAH associated with atrial septic defect (ASD), PAH associated with ventricular septic defect (VSD) and PAH associated with patent ductus arteriosus.

PAH associated with drugs or toxins shall be understood to encompass PAH associated with ingestion of aminorex, PAH associated with ingestion of a fenfluramine compound (e.g., PAH associated with ingestion of fenfluramine or PAH associated with ingestion of dexfenfluramine), PAH associated with ingestion of certain toxic oils (e.g., PAH associated with ingestion of rapeseed oil), PAH associated with ingestion of pyrrolizidine alkaloids (e.g., PAH associated with ingestion of bush tea) and PAH associated with ingestion of monocrotaline.

PAH associated with Other shall be understood to encompass PAH associated with a thyroid disorder, PAH associated with glycogen storage disease, PAH associated with Gaucher disease, PAH associated with hereditary hemorrhagic telangiectasia, PAH associated with a hemoglobinopathy, PAH associated with a myeloproliferative disorder, and PAH associated with splenectomy.

PAH associated with significant venous or capillary involvement shall be understood to encompass PAH associated with pulmonary veno-occlusive disease (PVOD) and PAH associated with pulmonary capillary hemangiomatosis (PCH).

Evidence for the association of PAH with scleroderma and the beneficial effect of an agonist of the PGI2 receptor on PAH is given by Badesch et al. (Badesch et al., Ann. Intern. Med., 2000, 132:425-434). Evidence for the association of PAH with the collagen vascular diseases mixed connective tissue disease (MCTD), systemic lupus ery thematosus (SLE), Sjogren's syndrome and CREST syndrome and the beneficial effect of an agonist of the PGI2 receptor on PAH is given by Humbert et al. (Eur. Respir. J., 1999, 13:1351-1356). Evidence for the association of PAH with CREST syndrome and the beneficial effect of an agonist of the PGI2 receptor on PAH is given by Miwa et al. (Int. Heart J., 2007, 48:417-422). Evidence for the association of PAH with SLE and the beneficial effect of an agonist of the PGI2 receptor on PAH is given by Robbins et al. (Chest, 2000, 117:14-18). Evidence for the association of PAH with HIV infection and the beneficial of an agonist of the PGI2 receptor on PAH is given by Aguilar et al. (Am. J. Respir. Crit. Care Med., 2000, 162:1846-1850). Evidence for the association of PAH with congenital heart defects (including ASD, VSD and patent ductus arteriosus) and the beneficial effect of an agonist of the PGI2 receptor on PAH is given by Rosenzweig et al. (Circulation, 1999, 99:1858-1865). Evidence for the association of PAH with fenfluramine and with dexfenfluramine, anorexigens, is given by Archer et al. (Am. J. Respir. Crit. Care Med., 1998, 158:1061-1067). Evidence for the association of PAH with hereditary hemorrhagic telangiectasia is given by McGoon et al (Chest, 2004, 126:14-34). Evidence for the association of PAH with splenectomy is given by Hoeper et al. (Ann. Intern. Med., 1999, 130:506-509). Evidence for the association of PAH with portal hypertension and the beneficial effect of an agonist of the PGI2 receptor on PAH is given by Hoeper et al. (Eur. Respir. J., 2005, 25:502-508).

Symptoms of PAH include dyspnea, angina, syncope and edema (McLaughlin et al., Circulation, 2006, 114:1417-1431). The compounds disclosed herein are useful in the treatment of symptoms of PAH.

In some embodiments, pulmonary arterial hypertension (PAH) is selected from: idiopathic PAH; familial PAH; PAH associated with a collagen vascular disease selected from: scleroderma, CREST syndrome, systemic lupus erythematosus (SLE), rheumatoid arthritis. Takayasu's arteritis, polymyositis, and dermatomyositis; PAH associated with a congenital heart disease selected from: atrial septic defect (ASD), ventricular septic defect (VSD) and patent ductus arteriosus in a patient; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); and PAH associated with pulmonary capillary hemangiomatosis (PCH) in a patient.

Studies indicate that patients with PAH have alterations in prostacyclin and thromboxane A2 activity, increased endothelin synthesis, and reduction in the expression of nitric oxide synthase in the pulmonary arterial system, all of which contribute to vasoconstriction/vasodilation imbalance, thrombosis, cell proliferation, and remodeling of the pulmonary arterial walls. Currently available pharmacotherapy for PAH targets the prostacyclin (also termed prostaglandin I₂ [PGI₂]), endothelin, and nitric oxide pathways.

Treatment guidelines for PAH support the use of an oral endothelin receptor antagonist (ERA), a phosphodiesterase type 5 inhibitor (PDE5-I), or a soluble guanylate cyclase (sGC) stimulator as monotherapy or in combination in PAH patients with WHO/NHYA functional class III. ERAs target the endothelin pathway, whereas PDES-Is and sGC stimulators target the nitric oxide pathway. There are 3 commercially available ERAs, ambrisentan, bosentan, and macitentan; 2 commercially available PDE5-Is, sildenafil and tadalafil; and 1 sGC stimulator, riociguat, which are approved for treatment of PAH. These medications can improve exercise capacity, symptoms, and/or cardiopulmonary hemodynamic variables in patients with symptomatic PAH.

PGI₂ is a metabolite of arachidonic acid and is formed via the cyclo-oxygenase pathway. Endothelial cells are the main source of PGI₂. The vascular effects of PGI₂ and its mimetics are largely mediated by activation of the PGI₂ (IP) receptor, and include vasodilation, the inhibition of smooth muscle cell proliferation, and the inhibition of platelet aggregation. The IP receptor is expressed on platelets and on the smooth muscle cells of several tissues, including lung, heart, aorta, liver, kidney, and blood vessels. Activation of the IP receptor results in increased cellular cyclic adenosine monophosphate (CAMP) followed by vasodilation in arteries and inhibition of aggregation in platelets. Improved hemodynamics, exercise capacity, and survival have been clearly demonstrated for PGI₂ replacement therapies.

Epoprostenol, a synthetic PGI, analogue, is a potent vasodilator and inhibitor of platelet aggregation, and was the first targeted PAH therapy to be approved. Epoprostenol improves prognosis for patients with PAH compared to conventional therapy, supporting utility of the IP receptor as a target for PAH therapy. However, epoprostenol requires continuous infusion through a portable pump, is unstable at room temperature, and is associated with intravenous catheter-related infections and thrombosis. Per the treatment algorithm put forth by the 5th World Symposium as well as the European Society of Cardiology/European Respiratory Society (ESC/ERS) guidelines, injectable prostacyclin analogues should be administered in patients whose PAH severity is categorized as WHO/NYHA functional class III through IV.

Subsequent PGI₂ analogues, such as treprostinil (continuous subcutaneous and intravenous infusion, intermittent inhalation, and oral) and iloprost (intermittent inhalation), have demonstrated efficacy through improved exercise capacity and/or delay in clinical worsening. These prostacyclins are prescribed for patients with WHO/NYHA functional class II through IV PAH. Although these prostacyclin analogs address some of the limitations associated with epoprostenol, they too have drawbacks with respect to frequent dosing (iloprost) and injection site pain (subcutaneous treprostinil), in addition to typical prostacyclin-associated side effects, such as headache, nausea, flushing, diarrhea, and jaw pain.

Selexipag is an oral, selective IP receptor agonist that is approved in the US and elsewhere for the treatment of PAH to delay disease progression and reduce the risk of hospitalization for PAH. The ESC/ERS guidelines recommend using selexipag to treat patients with PAH whose severity is WHO/NYHA functional class II through III. Although selexipag and its active metabolite have modes of action similar to that of endogenous prostacyclin (IP receptor agonism), they are chemically distinct from prostacyclin analogues with different pharmacologic properties. Selexipag has been shown to reduce PVR after 17 weeks of treatment, and demonstrated a reduction in a composite morbidity and mortality endpoint by 40%. However, the short effective half-life of the active metabolite of selexipag (3 to 4 hours) leads to relatively large fluctuations between peak and trough plasma concentrations after BID administration.

Despite the number of treatments available, the functional limitation and survival of patients with PAH remains unsatisfactory. The success of selexipag in delaying disease progression and reducing the risk of hospitalization for PAH supports the utility of oral prostacyclin therapies and paves the way for further optimization of nonprostanoid IP receptor agonists Research efforts focus on optimizing nonprostanoid IP receptor activation, bioavailability, and PK aimed to provide unremitting and potent target engagement with an oral formulation that provides clinical efficacy similar to parenteral prostacyclins.

As described herein, the compounds herein may be an attractive oral alternative to the currently approved oral prostacyclin analogues and nonprostanoid IP receptor agonists to treat PAH.

In some embodiments, the compounds disclosed herein are useful in the treatment PH other than PAH. For example, the compounds disclosed herein may be useful for treating forms of Group 3 PH, such as PH-ILD or PH associated with pulmonary fibrosis.

The methods and compositions of the present disclosure can also be suitable for treating other conditions such as platelet aggregation; coronary artery disease; myocardial infarction; transient ischemic attack; angina; stroke; ischemia-reperfusion injury; restenosis; atrial fibrillation; blood clot formation in an angioplasty or coronary bypass surgery individual or in an individual suffering from atrial fibrillation; atherothrombosis; asthma or a symptom thereof; a diabetic-related disorder such as diabetic peripheral neuropathy, diabetic nephropathy or diabetic retinopathy; glaucoma or another disease of the eye with abnormal intraocular pressure; hypertension; inflammation; psoriasis; psoriatic arthritis; rheumatoid arthritis; Crohn's disease; transplant rejection; multiple sclerosis; systemic lupus ery thematosus (SLE); ulcerative colitis; atherosclerosis; acne; type 1 diabetes; type 2 diabetes; sepsis; and chronic obstructive pulmonary disorder (COPD).

In some embodiments, the methods and compositions of the present disclosure are useful for treating chronic thromboembolic pulmonary hypertension (CTEPH). In some embodiments, the methods and compositions disclosed herein are useful for treating persistent/recurrent CTEPH (WHO Group 4) after surgical treatment. In some embodiments, the methods and compositions disclosed herein are useful for treating inoperable CTEPH to improve exercise capacity and/or WHO functional class

### Other PGI2 Related Diseases and Conditions

Other PGI2 related diseases and conditions include, but are not limited to antiplatelet therapies, atherosclerosis, asthma, diabetic-related pathologies, glaucoma, hypertension, and anti-inflammation therapies.

### Antiplatelet Therapies (Conditions Related to Platelet Aggregation)

Antiplatelet agents (antiplatelets) are prescribed for a variety of conditions. For example, in coronary artery disease they are used to help prevent myocardial infarction or stroke in patients who are at risk of developing obstructive blood clots (e.g., coronary thrombosis).

In a myocardial infarction (“MI” or “heart attack”), the heart muscle does not receive enough oxygen-rich blood as a result of a blockage in the coronary blood vessels. If taken while an attack is in progress or immediately afterward (preferably within 30 min), antiplatelets can reduce the damage to the heart.

A transient ischemic attack (“TIA” or “mini-stroke”) is a brief interruption of oxygen flow to the brain due to decreased blood flow through arteries, usually due to an obstructing blood clot. Antiplatelet drugs have been found to be effective in preventing TIAs.

Angina is a temporary and often recurring chest pain, pressure or discomfort caused by inadequate oxygen-rich blood flow (ischemia) to some parts of the heart. In patients with angina, antiplatelet therapy can reduce the effects of angina and the risk of myocardial infarction.

Stroke is an event in which the brain does not receive enough oxygen-rich blood, usually due to blockage of a cerebral blood vessel by a blood clot. In high-risk patients, taking antiplatelets regularly has been found to prevent the formation of blood clots that cause first or second strokes.

Angioplasty is a catheter based technique used to open arteries obstructed by a blood clot. Whether or not stenting is performed immediately after this procedure to keep the artery open, antiplatelets can reduce the risk of forming additional blood clots following the procedure(s).

Coronary bypass surgery is a surgical procedure in which an artery or vein is taken from elsewhere in the body and grafted to a blocked coronary artery, rerouting blood around the blockage and through the newly attached vessel. After the procedure, antiplatelets can reduce the risk of secondary blood clots.

Atrial fibrillation is the most common type of sustained irregular heart rhythm (arrhythmia). Atrial fibrillation affects about two million Americans every year. In atrial fibrillation, the atria (the heart's upper chambers) rapidly fire electrical signals that cause them to quiver rather than contract normally. The result is an abnormally fast and highly irregular heartbeat. When given after an episode of atrial fibrillation, antiplatelets can reduce the risk of blood clots forming in the heart and traveling to the brain (embolism).

There is evidence that a PGI2 receptor agonist will inhibit platelet aggregation and thus be a potential treatment as an antiplatelet therapy (see, e.g., Moncada et al., Lancet, 1977, 1:18-20). It has been shown that genetic deficiency of the PGI2 receptor in mice leads to an increased propensity towards thrombosis (Murata et al., Nature, 1997, 388:678-682).

PGI2 receptor agonists can be used to treat, for example, claudication or peripheral artery disease as well as cardiovascular complications, arterial thrombosis, atherosclerosis, vasoconstriction caused by serotonin, ischemia-reperfusion injury, and restenosis of arteries following angioplasty or stent placement. (See, e.g., Fetalvero et al., Prostaglandins Other Lipid Mediat., 2007, 82:109-118; Archart et al., Curr. Med. Chem., 2007, 14: 2161-2169; Davi et al., N. Engl J. Med., 2007, 357:2482-2494; Fetalvero et al., Am. J. Physiol. Heart. Circ. Physiol., 2006, 290:H1337-H1346; Murata et al., Nature, 1997, 388:678-682; Wang et al., Proc. Natl. Acad. Sci. USA, 2006, 103:14507-14512; Xiao et al., Circulation, 2001, 104:2210-2215; McCormick et al., Biochem. Soc. Trans., 2007, 35:910-911; Arehart et al., Circ. Res., 2008, March 6 Epub ahead of print.)

PGI2 receptor agonists can also be used alone or in combination with thrombolytic therapy, for example, tissue-type plasminogen activator (t-PA), to provide cardioprotection following MI or postischemic myocardial dysfunction or protection from ischemic injury during percutaneous coronary intervention, and the like, including complications resulting therefrom. PGI2 receptor agonists can also be used in antiplatelet therapies in combination with, for example, alpha-tocopherol (vitamin E), echistatin (a disintegrin) or, in states of hypercoaguability, heparin. (See, e.g., Chan., J. Nutr., 1998, 128:1593-1596; Mardla et al., Platelets, 2004, 15:319-324; Bernabei et al., Ann. Thorac. Surg., 1995, 59:149-153; Gainza et al., J. Nephrol., 2006, 19:648-655.)

The PGI2 receptor agonists disclosed herein can provide beneficial improvement in microcirculation to patients in need of antiplatelet therapy by antagonizing the vasoconstrictive products of the aggregating platelets in, for example and not limited to the indications described above. Accordingly, in some embodiments, the present disclosure provides methods for reducing platelet aggregation in a patient in need thereof, comprising administering to the patient a composition comprising a PGI2 receptor agonist disclosed herein. In further embodiments, the present disclosure provides methods for treating coronary artery disease, myocardial infarction, transient ischemic attack, angina, stroke, atrial fibrillation, or a symptom of any of the foregoing in a patient in need of the treatment, comprising administering to the patient a composition comprising a PGI2 receptor agonist disclosed herein.

In further embodiments, the present disclosure provides methods for reducing risk of blood clot formation in an angioplasty or coronary bypass surgery patient, or a patient suffering from atrial fibrillation, comprising administering to the patient a composition comprising a PGI2 receptor agonist disclosed herein at a time where such risk exists.

### Atherosclerosis

Atherosclerosis is a complex disease characterized by inflammation, lipid accumulation, cell death and fibrosis. It is the leading cause of mortality in many countries, including the United States. Atherosclerosis, as the term is used herein, shall be understood to encompass disorders of large and medium-sized arteries that result in the progressive accumulation within the intima of smooth muscle cells and lipids.

It has been shown that an agonist of the PGI2 receptor can confer protection from atherosclerosis, such as from atherothrombosis (Arehart et al., Curr. Med. Chem., 2007, 14:2161-2169; Stitham et al., Prostaglandins Other Lipid Mediat., 2007, 82:95-108; Fries et al., Hematology Am. Soc. Hematol. Educ. Program, 2005, 445-451; Egan et al., Science, 2004, 306:1954-1957; Kobayashi et al., J. Clin. Invest., 2004, 114:784-794; Arehart et al., Circ. Res., 2008 Mar. 6 Epub ahead of print).

It has been shown that defective PGI2 receptor signaling appears to accelerate atherothrombosis in humans, i.e, that an agonist of the PGI2 receptor can confer protection from atherothrombosis in humans (Arehart et al., Circ. Res., 2008 Mar. 6 Epub ahead of print).

The compounds of the present disclosure can be useful in the treatment of atherosclerosis, and the treatment of the symptoms thereof. Accordingly, in some embodiments, the present disclosure provides methods for treating atherosclerosis in a patient in need of the treatment, comprising administering to the patient a composition comprising a PGI2 receptor agonist disclosed herein. In further embodiments, methods are provided for treating a symptom of atherosclerosis in a patient in need of the treatment, comprising administering to the patient a composition comprising a PGI2 receptor agonist disclosed herein.

### Asthma

Asthma is a lymphocyte-mediated inflammatory airway disorder characterized by airway eosinophilia, increased mucus production by goblet cells, and structural remodeling of the airway wall. The prevalence of asthma has dramatically increased worldwide in recent decades. It has been shown that genetic deficiency of the PGI2 receptor in mice augments allergic airway inflammation (Takahashi et al., Br J Pharmacol, 2002, 137:315-322). It has been shown that an agonist of the PGI2 receptor can suppress not only the development of asthma when given during the sensitization phase, but also the cardinal features of experimental asthma when given during the challenge phase (Idzko et al., J. Clin. Invest., 2007, 117:464-472; Nagao et al., Am. J. Respir. Cell Mol. Biol., 2003, 29:314-320), at least in part through markedly interfering with the function of antigen-presenting dendritic cells within the airways (Idzko et al., J. Clin. Invest., 2007, 117:464-472; Thou et Immunol., 2007, 178:702-710; Jaffar et al., J. Immunol., 2007, 179:6193-6203; Jozefowski et al., Int. Immunopharmacol., 2003, 3:865-878). These cells are crucial for both the initiation and the maintenance phases of allergic asthma, as depletion of airway dendritic cells during secondary challenge in sensitized mice abolished all characteristic features of asthma, an effect that could be completely restored by adoptive transfer of wild-type dendritic cells (van Rijt et al., J. Exp. Med., 2005, 201:981-991). It has also been shown that an agonist of the PGI2 receptor can inhibit proinflammatory cytokine secretion by human alveolar macrophages (Raychaudhuri et al., J. Biol. Chem., 2002, 277:33344-33348). The compounds of the present disclosure can be useful in the treatment of asthma, and the treatment of the symptoms thereof. Accordingly, in some embodiments, the present disclosure provides methods for treating asthma in a patient in need of the treatment, comprising administering to the patient a composition comprising a PGI2 receptor agonist disclosed herein. In further embodiments, methods are provided for treating a symptom of asthma in a patient in need of the treatment, comprising administering to the patient a composition comprising a PGI2 receptor agonist disclosed herein.

### Diabetic-Related Pathologies

Although hyperglycemia is the major cause for the pathogenesis of diabetic complications such as diabetic peripheral neuropathy (DPN), diabetic nephropathy (DN) and diabetic retinopathy (DR), enhanced vasoconstriction and platelet aggregation in diabetic patients has also been implicated to play a role in disease progression (Cameron et al., Naunyn Schmiedebergs Arch. Pharmacol., 2003, 367:607-614). Agonists of the PGI2 receptor promote vasodilation and inhibit platelet aggregation. Improving microvascular blood flow is able to benefit diabetic complications (Cameron, Diabetologia, 2001, 44:1973-1988).

It has been shown that an agonist of the PGI2 receptor can prevent and reverse motor and sensory peripheral nerve conduction abnormalities in streptozotocin-diabetic rats (Cotter et Naunyn Schmiedebergs Arch. Pharmacol., 1993, 347:534-540). Further evidence for the beneficial effect of an agonist of the PGI2 receptor in the treatment of diabetic peripheral neuropathy is given by Hotta et al. (Diabetes, 1996, 45:361-366), Ueno et al. (Jpn. J. Pharmacol., 1996, 70:177-182), Ueno et al. (Life Sci., 1996, 59:PL105-PL110), Hotta et al. (Prostaglandins, 1995, 49:339-349), Shindo et al. (Prostaglandins, 1991, 41:85-96), Okuda et al. (Prostaglandins, 1996, 52:375-384), and Koike et al (FASEB J., 2003, 17:779-781). Evidence for the beneficial effect of an agonist of the PGI2 receptor in the treatment of diabetic nephropathy is given by Owada et al. (Nephron, 2002, 92:788-796) and Yamashita et al. (Diabetes Res. Clin. Pract., 2002, 57:149-161). Evidence for the beneficial effect of an agonist of the PGI2 receptor in the treatment of diabetic retinopathy is given by Yamagishi et al. (Mol. Med., 2002, 8:546-550) , Burnette et al. (Exp. Eye Res., 2006, 83:1359-1365), and Hotta et al. (Diabetes. 1996, 45:361-366). It has been shown that an agonist of the PGI2 receptor can reduce increased tumor necrosis factor-α (TNF-α) levels in diabetic patients, implying that an agonist of the PGI2 receptor may contribute to the prevention of progression in diabetic complications (Fujiwara et al., Exp. Clin. Endocrinol. Diabetes, 2004, 112:390-394).

### Glaucoma

Evidence that topical administration of an agonist of the PGI2 receptor can result in a decrease in intraocular pressure (IOP) in rabbits and dogs and thereby have beneficial effect in the treatment of glaucoma is given by Hoyng et al. (Hoyng et al., Invest. Ophthalmol. Vis. Sci., 1987, 28:470-476).

### Hypertension

Agonists of the PGI2 receptor have been shown to have activity for regulation of vascular tone, for vasodilation, and for amelioration of pulmonary hypertension (see, e.g., Strauss et al., Clin Chest Med. 2007, 28:127-142; Driscoll et al., Expert Opin. Pharmacother., 2008, 9:65-81). Evidence for a beneficial effect of an agonist of the PGI2 receptor in the treatment of hypertension is given by Yamada et al. (Peptides, 2008, 29:412-418). Evidence that an agonist of the PGI2 receptor can protect against cerebral ischemia is given by Dogan et al. (Gen. Pharmacol., 1996, 27:1163-1166) and Fang et al. (J. Cereb. Blood Flow Metab., 2006, 26:491-501).

### Anti-Inflammation Therapies

Anti-inflammation agents are prescribed for a variety of conditions. For example, in an inflammatory disease they are used to interfere with and thereby reduce an underlying deleterious There is evidence that a PGI2 receptor agonist can inhibit inflammation and thus be a potential treatment as an anti-inflammation therapy. It has been shown that an agonist of the PGI2 receptor can inhibit pro-inflammatory cytokine and chemokine (interleukin-12 (IL-12), tumor necrosis factor-α (TNF-α), IL-1α, IL-6, macrophage inflammatory protein-1alpha (MIP-1α), monocyte chemoattractant protein-1 (MCP-1)) production and T cell stimulatory function of dendritic cells (Jozefowski et al., Int. Immunopharmacol., 2003, 865-878; Thou et al., J. Immunol., 2007, 178:702-710; Nagao et al., Am. J. Respir. Cell Mol. Biol., 2003, 29:314-320; Idzko et al., J. Clin. Invest., 2007, 117:464-472). It has been shown that an agonist of the PGI2 receptor can inhibit pro-inflammatory cytokine (TNF-α, IL-1β, IL-6, granulocyte macrophage stimulating factor (GM-CSF)) production by macrophages (Raychaudhuri et al., J. Biol. Chem., 2002, 277:33344-33348; Czeslick et al., Eur. J. Clin. Invest., 2003, 33:1013-1017; Di Renzo et al., Prostaglandin Leukot. Essent. Fatty Acids, 2005, 73:405-410; Shinomiya et al., Biochem. Pharmacol., 2001, 61:1153-1160). It has been shown that an agonist of the PGI2 receptor can stimulate anti-inflammatory cytokine (IL-10) production by dendritic cells (Jozefowski et al., Int. Immunopharmacol., 2003, 865-878; Zhou et al., J. Immunol., 2007, 178:702-710). It has been shown that an agonist of the PGI2 receptor can stimulate anti-inflammatory cytokine (IL-10) production by macrophages (Shinomiya et al., Biochem. Pharmacol., 2001, 61:1153-1160). It has been shown that an agonist of the PGI2 receptor can inhibit a chemokine (CCL17)-induced chemotaxis of leukocytes (CD4+ Th2 T cells) (Jaffar et al., J. Immunol., 2007, 179:6193-6203). It has been shown that an agonist of the PGI2 receptor can confer protection from atherosclerosis, such as from atherothrombosis (Arehart et al., Curr. Med. Chem., 2007, 14:2161-2169; Stitham et al., Prostaglandins Other Lipid Mediat, 2007, 82:95-108; Fries et al., Hematology Am. Soc. Hematol. Educ. Program. 2005, :445-451; Egan et al., Science, 2004, 306:1954-1957; Kobayashi et al., J. Clin. Invest., 2004, 114:784-794; Archart et al., Circ. Res., 2008 Mar. 6 Epub ahead of print). It has been shown that an agonist of the PGI2 receptor can attenuate asthma (Idzko et al., J. Clin. Invest., 2007, 117:464-472; Jaffar et al., J. Immunol., 2007, 179:6193-6203; Nagao et al., Am. J. Respir. Cell. Mol. Biol., 2003, 29:314-320). It has been shown that an agonist of the PGI2 receptor can decrease TNF-α production in type 2 diabetes patients (Fujiwara et al., Exp. Clin. Endocrinol. Diabetes, 2004, 112:390-394; Goya et al., Metabolism, 2003, 52:192-198). It has been shown that an agonist of the PGI2 receptor can inhibit ischemia-reperfusion injury (Xiao et al., Circulation, 2001, 104:2210-2215). It has been shown that an agonist of the PGI2 receptor can inhibit restenosis (Cheng et al., Science, 2002, 296:539-541). It has been shown that an agonist of the PGI2 receptor can attenuate pulmonary vascular injury and shock in a rat model of septic shock (Harada et al., Shock, 2008 Feb. 21 Epub ahead of print). It has been shown that an agonist of the PGI2 receptor can reduce the serum levels of TNF-α in vivo in patients with rheumatoid arthritis, and this is associated with improvement in the clinical course of the disease (Gao et al., Rheumatol. Int., 2002, 22:45-51; Boehme et al., Rheumatol. Int., 2006, 26:340-347).

The compounds of the present disclosure can provide beneficial reduction of inflammation. The compounds of the present disclosure can provide beneficial reduction of a deleterious inflammatory response associated with an inflammatory disease. Accordingly, in some embodiments, the present disclosure provides methods for reducing inflammation in a patient in need thereof, comprising administering to the patient a composition comprising a PGI2 receptor agonist disclosed herein. In some embodiments, the present disclosure provides methods for decreasing IL-12, TNF-α, IL-1α, IL-1β, IL-6, MIP-1α or MCP-1 production in a patient in need thereof, comprising administering to the patient a composition comprising a PGI2 receptor agonist disclosed herein. In some embodiments, the present disclosure provides methods for decreasing TNF-α production in a patient in need thereof, comprising administering to the patient a composition comprising a PGI2 receptor agonist disclosed herein. In some embodiments, the present disclosure provides methods for increasing IL-10 production in a patient in need thereof, comprising administering to the patient a composition comprising a PGI2 receptor agonist disclosed herein. In some embodiments, the present disclosure provides methods for reducing a deleterious inflammatory response associated with an inflammatory disease in a patient in need thereof, comprising administering to the patient a composition comprising a PGI2 receptor agonist disclosed herein. In some embodiments, the present disclosure provides methods for treating an inflammatory disease or a symptom thereof in a patient in need of the treatment comprising administering to the patient a composition comprising a PGI2 receptor agonist disclosed herein. In some embodiments, the present disclosure provides methods for treating an inflammatory disease or a symptom thereof in a patient in need of the treatment comprising administering to the patient a composition comprising a PGI2 receptor agonist disclosed herein. In some embodiments, the present disclosure provides methods for treating an inflammatory disease or a symptom thereof in a patient in need of the treatment comprising administering to the patient a composition comprising a PGI2 receptor agonist disclosed herein, wherein the inflammatory disease is selected from the group consisting of psoriasis, psoriatic arthritis, rheumatoid arthritis, Crohn's disease, transplant rejection, multiple sclerosis, systemic lupus ery thematosus (SLE), ulcerative colitis, ischemia-reperfusion injury, restenosis, atherosclerosis, acne, diabetes (including type 1 diabetes and type 2 diabetes), sepsis, chronic obstructive pulmonary disease (COPD), and asthma.

In one aspect, described herein are methods of treating a disease or condition associated with a PGI2 receptor. In some embodiments, the disease or condition is PAH. In some embodiments, the disease or condition is selected from: pulmonary arterial hypertension (PAH); idiopathic PAH; familial PAH; PAH associated with a collagen vascular disease, a congenital heart disease, portal hypertension, HIV infection, ingestion of a drug or toxin, hereditary hemorrhagic telangiectasia, splenectomy, pulmonary veno-occlusive disease (PVOD) or pulmonary capillary hemangiomatosis (PCH); PAH with significant venous or capillary involvement; platelet aggregation; coronary artery disease; myocardial infarction; transient ischemie attack; angina; stroke; ischemia-reperfusion injury; restenosis; atrial fibrillation; blood clot formation in an angioplasty or coronary bypass surgery individual or in an individual suffering from atrial fibrillation; atherosclerosis; atherothrombosis; asthma or a symptom thereof; a diabetic-related disorder such as diabetic peripheral neuropathy, diabetic nephropathy or diabetic retinopathy; glaucoma or other disease of the eye with abnormal intraocular pressure; hypertension; inflammation; psoriasis; psoriatic arthritis; rheumatoid arthritis; Crohn's disease; transplant rejection; multiple sclerosis; systemic lupus erythematosus (SLE); ulcerative colitis; ischemia-reperfusion injury; restenosis; atherosclerosis; acne; type 1 diabetes; type 2 diabetes; sepsis; and chronic obstructive pulmonary disorder (COPD).

Disclosed herein are methods of treating pulmonary arteril hypertension (PAH) in a subject in need thereof, comprising administering to the subject a therapeutically effective amount of a compound disclosed herein, or a pharmaceutically acceptable salt or solvate thereof, or a pharmaceutical composition disclosed herein.

The methods described herein can be administered to a subject with any suitable symptoms known by one of skill in the art. In some embodiments, the subject has one or more World Health Organization (WHO)/New York Heart Association (NYHA) Functional class (FC) II to III symptoms. In some embodiments, the symptoms include, but are not limited to slight limitations of physical activity, being comfortable at rest, and/or ordinary physical activity causes undue dyspnea or fatigue, chest pain, or near syncope. In some embodiments, the symptoms include, but are not limited to marked limitation of physical activity, being comfortable at rest, and/or ordinary activity causes undue dyspnea or fatigue, chest pain, or near syncope.

Pulmonary arteril hypertension (PAH) can be any pulmonary arteril hypertension known by one of skill in the art. In some embodiments, the PAH is selected from: idiopathic PAH; familial PAH; PAH associated with a collagen vascular disease selected from: scleroderma, CREST syndrome, systemic lupus erythematosus (SLE), rheumatoid arthritis. Takayasu's arteritis, polymyositis, and dermatomyositis; PAH associated with a congenital heart disease selected from: atrial septic defect (ASD), ventricular septic defect (VSD) and patent ductus arteriosus in an individual; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); and PAH associated with pulmonary capillary hemangiomatosis (PCH) in an individual. In some embodiments, the PAH is heritable pulmonary arterial hypertension (HPAH), familial PAH, simplex PAH, or familial primary pulmonary hypertension. In some embodiments, the PAH is familial primary pulmonary hypertension.

In one aspect, disclosed berein are methods of modulating a prostacyclin (PGI2) receptor in a subject in need thereof, comprising administering to the subject a therapeutically effective amount of a compound disclosed herein, or a pharmaetucially acceptable salt or solvate thereof, or a pharmaceutical composition disclosed herein.

In one aspect, disclosed herein are methods of treating a disease or condition associated with prostacyclin (PGI2) receptor in a subject in need thereof, comprising administering to the subject a therapeutically effective amount of a compound disclosed herein, or a pharmaceutically acceptable salt or solvate thereof, or a pharmaceutical composition disclosed herein.

Diseases or conditions associated with PGI2 receptor can be any disease known by one of skill in the art. In some embodiments, the disease or condition is selected from inflammatory diseases. In some embodiments, the disease or condition is selected from allergic inflammation, cytokine-mediated inflammation, emphysema, fibrosis, angina infarction, myocardial infarction, pulmonary arterial hypertension (PAH), pulmonary hypertension, hypertension, connective tissue diseases, vascular diseases, cardiovascular diseases, lung diseases, and respiratory tract disease. In some embodiments, the disease or condition is selected from angina infarction, myocardial infarction, pulmonary arterial hypertension (PAH), pulmonary hypertension, hypertension, connective tissue diseases, vascular diseases, cardiovascular diseases, lung diseases, and respiratory tract disease. In some embodiments, the disease or condition is selected from pulmonary arterial hypertension (PAH), pulmonary hypertension, hypertension, connective tissue discases, vascular diseases, cardiovascular diseases, lung diseases, and respiratory tract disease. In some embodiments, the disease or condition is selected from ulmonary arterial hypertension (PAH), pumlmonary hypertension, hypertension, or cardiovascular diseases.

The compound administered in the methods described herein can be administered by an suitable method known by one of skill in the art. In some embodiments, the compound is administered via a titration scheme.

In some embodiments, the compound is administered once daily. In some embodiments, the compound is administered twice daily. In some embodiments, the compound is administered in an amount as described herein. In some embodiments, the compound is administered in the amont of about 0.01 mg to about 10 mg per day. In some embodiments, the compound is administered in the amount of about 0.01 mg to about 5 mg per day. In some embodiments, the compound is administered in an amount of about 0.01 mg to about 2 mg per day. In some embodiments, the compound is administered in an amount of about 0.05 mg to about 1.5 mg per day. In some embodiments, the compound is administered in an amount of about 0.05 mg to about 1.2 mg per day.

In one aspect, disclosed herein is the use of a compound disclosed herein, or a pharmaceutically acceptable salt or solvate thereof, or a pharmaceutical composition described herein, in the manufacture of a medicament for the treatment of pulmonary arterial hypertension (PAH).

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”).

Once improvement of the patient's conditions has occurred, a maintenance dose is administered if necessary. Subsequently, in specific embodiments, the dosage or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

The dose when using the compounds of the present disclosure can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present disclosure. Representative doses of the present disclosure include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present disclosure and as part of a drug combination. The dosage regimen for treating a disease condition with the compounds and/or compositions of this disclosure is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this disclosure.

The desired dose may conveniently be presented in a single dose or as divided doses administered at appropriate intervals, for example, as two, three, four or more sub-doses per day The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

Compounds of the present disclosure can be administrated in a wide variety of oral and parenteral dosage forms. It will be obvious to those skilled in the art that the following dosage forms may comprise, as the active component, either a compound of the disclosure or a pharmaceutically acceptable salt, solvate or hydrate of a compound of the disclosure.

In certain embodiments, the compositions containing the compounds and solid state forms described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-0.6 mg per day. In one embodiment, the desired dose is conveniently presented in a single dose or in divided doses administered simultaneously or at appropriate intervals, for example as two or more sub-doses per day. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In any of the aforementioned aspects are further embodiments in which the effective amount of the compound disclosed herein, or a pharmaceutically acceptable salt thereof, is: (a) systemically administered to the mammal; and/or (b) administered orally to the mammal.

In some embodiments, the compound disclosed herein, or a pharmaceutically acceptable salt thereof, is administered at a dose that is about 0.01 mg, about 0.02 mg, about 0.03 mg, about 0.04 mg, about 0.05 mg, about 0.06 mg, about 0.07 mg, about 0.08 mg, about 0.09 mg, about 0. 1 mg, about 0.12 mg, about 0.13 mg, about 0.14 mg, about 0.15 mg, about 0.16 mg, about 0.17 mg, about 0.18 mg, about 0.19 mg, about 0.2 mg, about 0.21 mg, about 0.22 mg, about 0.23 mg, about 0.24 mg, about 0.25 mg, about 0.26 mg, about 0.27 mg, about 0.28 mg, about 0.29 mg, about 0.3 mg, about 0.31 mg, about 0.32 mg, about 0.33 mg, about 0.34 mg, about 0.35 mg, about 0.36 mg, about 0.37 mg, about 0.38 mg, about 0.39 mg, about 0.4 mg, about 0.41 mg, about 0.42 mg, about 0.43 mg, about 0.44 mg, about 0.45 mg, about 0.46 mg, about 0.47 mg, about 0.48 mg, about 0.49 mg, about 0.5 mg, about 0.51 mg, about 0.52 mg, about 0.53 mg, about 0.54 mg, about 0.55 mg, about 0.56 mg, about 0.57 mg, about 0.58 mg, about 0.59 mg, about 0.6 mg, about 0.8 mg, about 1.0 mg, about 1.2 mg, about 1.4 mg, about 1.6 mg, about 1.8 mg, about 2.0 mg, about 2.5 mg, or about 3 mg. In some embodiments, the compound disclosed herein, or a pharmaceutically acceptable salt thereof, is administered at a dose that is equivalent to about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.12 mg, about 0.13 mg, about 0.14 mg, about 0.15 mg, about 0.16 mg, about 0.17 mg, about 0.18 mg, about 0.19 mg, about 0.2 mg, about 0.21 mg, about 0.22 mg, about 0.23 mg, about 0.24 mg, about 0.25 mg, about 0.26 mg, about 0.27 mg, about 0.28 mg, about 0.29 mg, about 0.3 mg, about 0.31 mg, about 0.32 mg, about 0.33 mg, about 0.34 mg, about 0.35 mg, about 0.36 mg, about 0.37 mg, about 0.38 mg, about 0.39 mg, about 0.4 mg, about 0.41 mg, about 0.42 mg, about 0.43 mg, about 0.44 mg, about 0.45 mg, about 0.46 mg, about 0.47 mg, about 0.48 mg, about 0.49 mg, about 0.5 mg, about 0.51 mg, about 0.52 mg, about 0.53 mg, about 0.54 mg, about 0.55 mg, about 0.56 mg, about 0.57 mg, about 0.58 mg, about 0.59 mg, about 0.6 mg, about 0.8 mg, about 1.0 mg, about 1.2 mg, about 1.4 mg, about 1.6 mg, about 1.8 mg, about 2.0 mg, about 2.5 mg, or about 3 mg of Compound 1. In some embodiments, the dose is administered once a day. In some embodiments, the dose is administered twice a day.

In some embodiments, the compound disclosed herein, or a pharmaceutically acceptable salt thereof, is administered at a dose that is about 0.1 mg, about 0.12 mg, about 0.13 mg, about 0.14 mg, about 0 15 mg, about 0.16 mg, about 0.17 mg, about 0.18 mg, about 0.19 mg, about 0.2 mg, about 0.3 mg, about 0.40 mg, about 0.42 mg, about 0.45 mg, about 0.48 mg, about 0.5 mg, about about 0.53 mg, about 0.55 mg, about 0.58 mg, about 0.6 mg, about 0.8 mg, about 1.0 mg, about 1.2 mg, about 1.4 mg, about 1.6 mg, about 1.8 mg, about 2.0 mg, about 2.5 mg, or about 3 mg. In some embodiments, the compound disclosed herein, or a pharmaceutically acceptable salt thereof, is administered at a dose that is equivalent to about 0.1 mg, about 0.12 mg, about 0.13 mg, about 0.14 mg, about 0.15 mg, about 0.16 mg, about 0.17 mg, about 0.18 mg, about 0.19 mg, about 0.2 mg, about 0.3 mg, about 0.40 mg, about 0.42 mg, about 0.45 mg, about 0.48 mg, about 0.5 mg, about about 0.53 mg, about 0.55 mg, about 0.58 mg, about 0.6 mg, about 0.8 mg, about 1.0 mg, about 1.2 mg, about 1.4 mg, about 1.6 mg, about 1.8 mg, about 2.0 mg, about 2.5 mg, or about 3 mg of Compound 1. In some embodiments, the compound disclosed herein, or a pharmaceutically acceptable salt thereof, is administered at a dose that is about 0.1 mg, about 0.2 mg, about 0.3 mg, about 0.4 mg, about 0.5 mg, about 0.6 mg, about 0.7 mg, about 0.8 mg, about 1.0 mg, about 1.2 mg, about 1.4 mg, about 1.6 mg, about 1.8 mg, or about 2.0 mg. In some embodiments, the compound disclosed herein, or a pharmaceutically acceptable salt thereof, is administered at a dose that is equivalent to about 0.1 mg, about 0.2 mg, about 0.3 mg, about 0.4 mg, about 0.5 mg, about 0.6 mg, about 0.7 mg, about 0.8 mg, about 1.0 mg, about 1.2 mg, about 1.4 mg, about 1.6 mg, about 1.8 mg, or about 2.0 mg of Compound 1. In some embodiments, the dose is administered once a day. In some embodiments, the dose is administered twice a day.

In some embodiments, the compound disclosed herein, or a pharmaceutically acceptable salt thereof, is administered at 0.05 mg per day. In some embodiments, the compound disclosed herein, or a pharmaceutically acceptable salt thereof, is administered at 0.10 mg per day In some embodiments, the compound disclosed herein, or a pharmaceutically acceptable salt thereof, is administered at 0.15 mg per day. In some embodiments, the compound disclosed herein, or a pharmaceutically acceptable salt thereof, is administered at 0.20 mg per day. In some embodiments, the compound disclosed herein, or a pharmaceutically acceptable salt thereof, is administered at 0.25 mg per day. In some embodiments, the compound disclosed herein, or a pharmaceutically acceptable salt thereof, is administered at 0.30 mg per day. In some embodiments, the compound disclosed herein, or a pharmaceutically acceptable salt thereof, is administered at 0.35 mg per day. In some embodiments, the compound disclosed herein, or a pharmaceutically acceptable salt thereof, is administered at 0.40 mg per day. In some embodiments, the compound disclosed herein, or a pharmaceutically acceptable salt thereof, is administered at 0.45 mg per day. In some embodiments, the compound disclosed herein, or a pharmaceutically acceptable salt thereof, is administered at 0.5 mg per day. In some embodiments, the compound disclosed herein, or a pharmaceutically acceptable salt thereof, is administered at 0.55 mg per day. In some embodiments, the compound disclosed herein, or a pharmaceutically acceptable salt thereof, is administered at 0.60 mg per day. In some embodiments, the compound disclosed herein, or a pharmaceutically acceptable salt thereof, is administered at 0.65 mg per day. In some embodiments, the compound disclosed herein, or a pharmaceutically acceptable salt thereof, is administered at 0.70 mg per day. In some embodiments, the compound disclosed herein, or a pharmaceutically acceptable salt thereof, is administered at 0.75 mg per day. In some embodiments, the compound disclosed herein, or a pharmaceutically acceptable salt thereof, is administered at 0.80 mg per day. In some embodiments, the compound disclosed herein, or a pharmaceutically acceptable salt thereof, is administered at 0.85 mg per day. In some embodiments, the compound disclosed herein, or a pharmaceutically acceptable salt thereof, is administered at 0.9 mg per day. In some embodiments, the compound disclosed herein, or a pharmaceutically acceptable salt thereof, is administered at 0.95 mg per day. In some embodiments, the compound disclosed herein, or a pharmaceutically acceptable salt thereof, is administered at 1 mg per day.

In some embodiments, the compound disclosed herein, or a pharmaceutically acceptable salt thereof, is administered at 0.05 mg per day, at 0.10 mg per day, at 0.15 mg per day, at 0.20 mg per day, at 0.25 mg per day, at 0.30 mg per day, at 0.35 mg per day, at 0.40 mg per day, at 0.45 mg per day, at 0.5 mg per day, at 0.55 mg per day, at 0.60 mg per day, at 0.65 mg per day, at 0.70 mg per day, at 0.75 mg per day, at 0.80 mg per day, at 0.85 mg per day, at 0.9 mg per day, at 0.95 mg per day, at 1 mg per day, at 1.2 mg per day, at 1.5 mg per day, at 2.0 mg per day, at 2.5 mg per day, or at 3.0 mg per day. In some embodiments, the compound disclosed herein, or a pharmaceutically acceptable salt thereof, is administered at at 0.5 mg per day, at 0.55 mg per day, at 0.60 mg per day, at 0.65 mg per day, at 0.70 mg per day, at 0.75 mg per day, at 0.80 mg per day, at 0.85 mg per day, at 0.9 mg per day, at 0.95 mg per day, at 1 mg per day, at 1.2 mg per day, at 1.5 mg per day, at 2.0 mg per day, at 2.5 mg per day, or at 3.0 mg per day In some embodiments, the compound disclosed herein, or a pharmaceutically acceptable salt thereof, is administered at at 0.9 mg per day, at 0.95 mg per day, at 1 mg per day, at 1.2 mg per day, at 1.5 mg per day, at 2.0 mg per day, at 2.5 mg per day, or at 3.0 mg per day.

In some embodiments, the compound disclosed herein, or a pharmaceutically acceptable salt thereof, is administered in an amount equivalent to about 0.05 mg of Compound 1 per day. In some embodiments, the compound disclosed herein, or a pharmaceutically acceptable salt thereof, is administered in an amount equivalent to about 0.1 mg of Compound 1 per day. In some embodiments, the compound disclosed herein, or a pharmaceutically acceptable salt thereof, is administered in an amount equivalent to about 0.15 mg of Compound 1 per day. In some embodiments, the compound disclosed herein, or a pharmaceutically acceptable salt thereof, is administered in an amount equivalent to about 0.2 mg of Compound 1 per day. In some embodiments, the compound disclosed herein, or a pharmaceutically acceptable salt thereof, is administered in an amount equivalent to about 0.25 mg of Compound 1 per day. In some embodiments, the compound disclosed herein, or a pharmaceutically acceptable salt thereof, is administered in an amount equivalent to about 0.3 mg of Compound 1 per day. In some embodiments, the compound disclosed herein, or a pharmaceutically acceptable salt thereof, is administered in an amount equivalent to about 0.35 mg of Compound 1 per day. In some embodiments, the compound disclosed herein, or a pharmaceutically acceptable salt thereof, is administered in an amount equivalent to about 0.4 mg of Compound 1 per day. In some embodiments, the compound disclosed herein, or a pharmaceutically acceptable salt thereof, is administered in an amount equivalent to about 0.45 mg of Compound 1 per day. In some embodiments, the compound disclosed herein, or a pharmaceutically acceptable salt thereof, is administered in an amount equivalent to about 0.5 mg of Compound 1 per day. In some embodiments, the compound disclosed herein, or a pharmaceutically acceptable salt thereof, is administered in an amount equivalent to about 0.55 mg of Compound 1 per day. In some embodiments, the compound disclosed herein, or a pharmaceutically acceptable salt thereof, is administered in an amount equivalent to about 0.6 mg of Compound 1 per day. In some embodiments, the compound disclosed herein, or a pharmaceutically acceptable salt thereof, is administered in an amount equivalent to about 0.65 mg of Compound 1 per day. In some embodiments, the compound disclosed herein, or a pharmaceutically acceptable salt thereof, is administered in an amount equivalent to about 0.7 mg of Compound 1 per day. In some embodiments, the compound disclosed herein, or a pharmaceutically acceptable salt thereof, is administered in an amount equivalent to about 0.75 mg of Compound 1 per day. In some embodiments, the compound disclosed herein, or a pharmaceutically acceptable salt thereof, is administered in an amount equivalent to about 0.8 mg of Compound 1 per day. In some embodiments, the compound disclosed herein, or a pharmaceutically acceptable salt thereof, is administered in an amount equivalent to about 0.85 mg of Compound 1 per day. In some embodiments, the compound disclosed herein, or a pharmaceutically acceptable salt thereof, is administered in an amount equivalent to about 0.9 mg of Compound 1 per day. In some embodiments, the compound disclosed hercin, or a pharmaceutically acceptable salt thereof, is administered in an amount equivalent to about 0.95 mg of Compound 1 per day. In some embodiments, the compound disclosed herein, or a pharmaceutically acceptable salt thereof, is administered in an amount equivalent to about 0.1 mg of Compound 1 per day.

In some embodiments, the dose is administered once a day. In some embodiments, the dose is administered twice a day.

In some embodiments, the compound disclosed herein, or a pharmaceutically acceptable salt thereof, is administered once a day. In some embodiments, the compound disclosed herein, or a pharmaceutically acceptable salt thereof, is administered twice a day. In some embodiments, the compound disclosed herein, or a pharmaceutically acceptable salt thereof, is administered 1 or 2 times a week. In some embodiments, the compound disclosed herein, or a pharmaceutically acceptable salt thereof, is administered every other day.

### Routes of Administration

Suitable routes of administration include, but are not limited to, oral, intravenous, aerosol, parenteral, pulmonary, transmucosal, transdermal, and topical administration. In addition, by way of example only, parenteral delivery includes intramuscular, subcutaneous, intravenous, intramedullary injections, as well as intrathecal, direct intraventricular, intraperitoneal, and intranasal injections.

In some embodiments, compounds of the present disclosure are administered orally once daily. In some embodiments, compounds of the present disclosure are administered orally twice daily.

In certain embodiments, a compound as described herein is administered in a local rather than systemic manner, for example, via injection of the compound directly into an organ, often in a depot preparation or sustained release formulation. In specific embodiments, Jong acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the disclosure in a suitable liquid vehicle and filter sterilizing the solution before filling and sealing an appropriate vial or ampule. These are just a few examples of the many appropriate methods well known in the art for preparing dosage forms.

Pharmaceutical formulations include those suitable for oral, topical (including buccal and sub-lingual), or parenteral (including intramuscular, subcutaneous and intravenous) administration or in a form suitable for administration by inhalation, insufflation or by a transdermal patch.

The compounds of the disclosure, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

### Pharmaceutical Compositions/Formulations

Described herein is a pharmaceutical composition, comprising a compound described herein, or a pharmaceutically acceptable salt or solvate thereof, and a pharmaceutically acceptable carrier or excipient. In some embodiments, the pharmaceutical composition is in a solid dosage form. In some embodiments, the pharmaceutical composition is a tablet or a capsule.

The compounds described herein are administered to a subject in need thereof, either alone or in combination with pharmaceutically acceptable carriers, excipients or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York. N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

For preparing pharmaceutical compositions from the compounds of the present disclosure. the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water. Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds according to the present disclosure may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the disclosure may be formulated as ointments, creams or lotions, or as a transdermal patch.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present disclosure or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present disclosure as an aerosol can be prepared by processes well known to the person skilled in the art. For their preparation, for example, solutions or dispersions of the compounds of the present disclosure in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

### Combination

Disclosed herein are methods of treating a disease or disorder associated with pulmonary hypertension (PH), pulmonary vascular resistance (PVR), pulmonary arterial hypertension (PAH), pulmonary hypertension associated with interstitial lung disease (PH-ILD), or a combination thereof, using a compound disclosed herein, or a pharmaceutically acceptable salt, solvate, or stercoisomer thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

### EXAMPLES

### Example 1: Synthesis of Ralinepag n-Butyl Ester (2a)

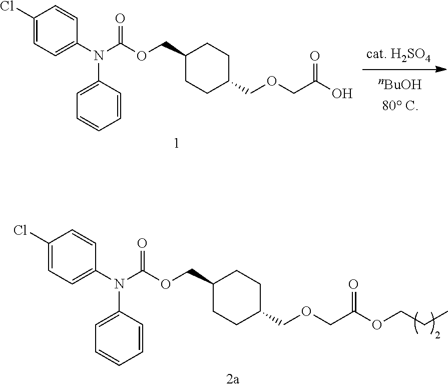

To the suspension of ralinepag acid (1) (0.21 g, 0.486 mmol) in n-butanol (5.0 mL) was added concentrated sulfuric acid (25 μL) at room temperature under argon. The resulting reaction mixture was heated at 80° C. for 3 h under argon. After 3 h, the reaction was found to be complete based on TLC. The solvent was removed in vacuo at 50° C., diluted with EtOAc (30 mL), dried over Na₂SO₄, filtered, and concentrated in vacuo to obtain crude ralinepag n-butyl ester (2a) (0.31 g). This was purified by silica gel column chromatography. The combined fractions were evaporated in vacuo and dried under high vacuum to give pure ralinepag n-butyl ester (2a) (0.23 g) in 96.2% yield as colorless viscous oil. The pure product was characterized by IR, ¹H NMR and LC-MS. HPLC purity of the product was found to be 99.14% and free of any ralinepag acid (1).

### Example 2: Synthesis of Ralinepag n-Dodecyl Ester (2b)

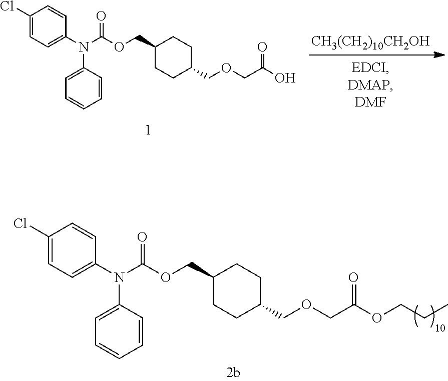

To a solution of ralinepag acid (1) (0.214 g, 0.495 mmol) in anhydrous DMF (2.0 mL) was added 1-dodecanol (0.093 g, 0.499 mmol) followed by EDCI·HCl (0.142 g, 0.741 mmol) and DMAP (0.181 g, 1.48 mmol) at room temperature under argon. The resulting reaction mixture was stirred overnight at room temperature under argon. After 22 h, the reaction was found to be complete based on TLC. The reaction mixture was diluted with EtOAc (15 mL) and treated with sat. aq. NH₄Cl (15 mL) and layers were separated. The organic layer was washed with sat. aq. NH₄Cl (10 mL), brine (10 mL), dried over Na₂SO₄, filtered, and concentrated in vacuo to obtain crude ralinepag n-dodecyl ester (2b) (0.18 g). This was purified by column chromatography. The combined fractions were evaporated in vacuo and dried under high vacuum to give pure ralinepag n-dodecyl ester (2b) (0.11 g) in 35.7% vield as waxy solid. The pure product was characterized by IR, ¹H NMR and LC-MS. HPLC purity of the product was found to be 96.68% and free of any ralinepag acid (1).

### Example 3: Synthesis of Ralinepag Cyclic Carbonate Ester (2c)

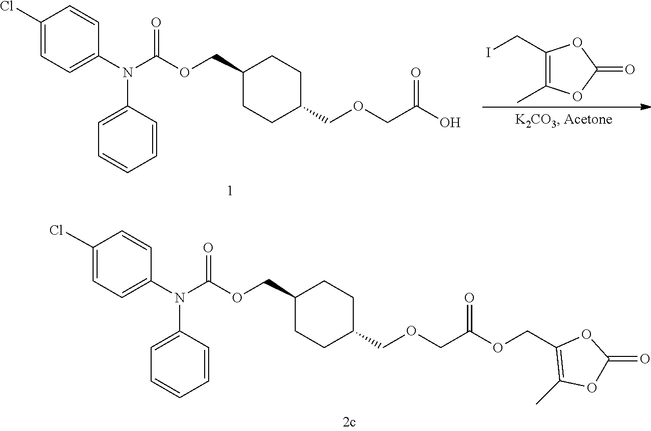

To a solution of ralinepag acid (1) (0.208 g, 0.48 mmol) in acetone (5.0 mL) was added a solution of 4-(iodomethyl)-5-methyl-1,3-dioxol-2-one (0.138 g, 0.58 mmol) in acetone (1.0 mL) followed by K₂CO₃ (0.200 g, 1.44 mmol) at room temperature under argon. The resulting reaction mixture was stirred overnight at room temperature under argon. After 23 h, the reaction was found to be complete based on TLC. The reaction mixture was filtered and concentrated in vacuo to obtain crude ralinepag cyclic carbonate ester (2c) (0.468 g). This was purified by silica gel column chromatography. The combined fractions were evaporated in vacuo and dried under high vacuum to give pure ralinepag cyclic carbonate ester (2c) (0.23 g) in 88.1% yield as clear viscous oil. The pure product was characterized by IR, ¹H NMR and LC-MS. HPLC purity of the product was found to be 96.29% and free of any ralinepag acid (1).

### Example 4: Synthesis of Ralinepag n-Octyl Ester (4a)

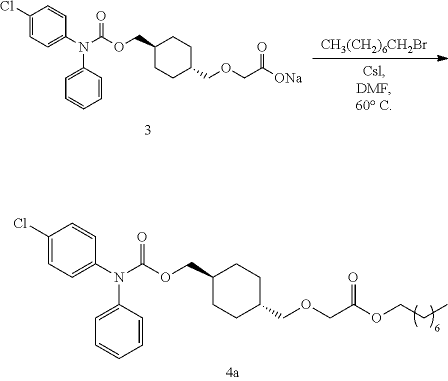

To a solution of ralinepag sodium salt (3) (0.251 g, 0.522 mmol) in anhydrous DMF (3.0 mL) was added cesium iodide (0.173 g, 0.666 mmol) followed by 1-bromooctane (0.19 mL, 1.103 mmol) at room temperature under argon. The resulting reaction mixture was at 60° C. for 3 h. After 3 h, the reaction was found to be complete based on TLC. The reaction mixture was cooled and was diluted with EtOAc (20 mL) and treated with sat. aq. NH₄Cl (20 mL) and layers were separated. The organic layer was washed with sat. aq. NH₄Cl (10 mL), water (20 mL), brine (5 mL), dried over Na₂SO₄, filtered, and concentrated in vacuo to obtain crude ralinepag n-octyl ester (4a) (0.90 g; combined with previous batch) This was purified by silica gel chromatography. The combined fractions were evaporated in vacuo and dried under high vacuum to give pure ralinepag n-octyl ester (4a) (0.21 g) in 49.8% yield as viscous oil. The pure product was characterized by IR, ¹H NMR and LC-MS. HPLC purity of the product was found to be 99.16% and free of any ralinepag acid (1).

### Example 5: Synthesis of Ralinepag tert-Butyl Glycolate Ester (4b)

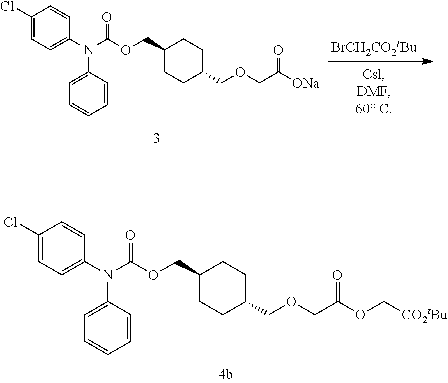

To a solution of ralinepag sodium salt (3) (0.50 g, 1.10 mmol) in anhydrous DMF (10.0 mL) was added cesium iodide (0.34 g, 1.32 mmol) followed by tert-butyl bromoacetate (0.82 mL, 5.50 mmol) at room temperature under argon. The resulting reaction mixture was at 60° C. for 6 h. After 6 h, the reaction was found to be complete based on TLC. The reaction mixture was cooled and was treated with sat. aq. NH₄Cl (30 mL) and extracted with EtOAc (3×30 mL). The organic layer was washed with brine (30 mL), dried over Na₂SO₄, filtered, and concentrated in vacuo to obtain crude ralinepag tert-butyl glycolate ester (4b) (5.3 g; combined with previous batch). This was purified by silica gel chromatography. The combined fractions were evaporated in vacuo and dried under high vacuum to give pure ralinepag tert-butyl glycolate ester (4b) (0.56 g) in 77.8% yield as viscous oil. The pure product was characterized by IR, ¹H NMR and LC-MS. HPLC purity of the product was found to be 99.17% and free of any ralinepag acid (1).

### Example 6: Synthesis of Ralinepag Methanesulfonamide (6)

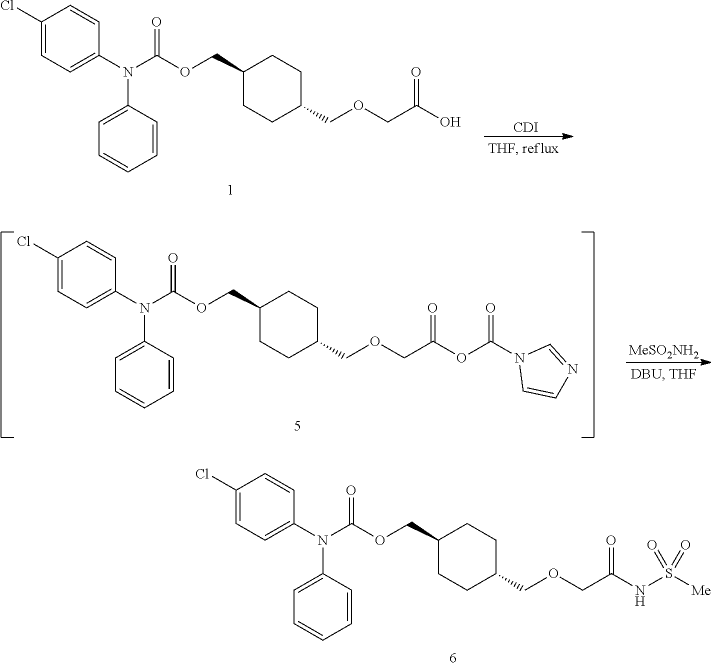

To a stirred solution of ralinepag acid (1) (0.20 g, 0.46 mmol) in THF (3.0 mL) was added CDI (0.11 g, 0.69 mmol) in one portion at room temperature under argon. The resulting reaction mixture was stirred for 1 h and then refluxed for 1 h under argon. The reaction mixture was cooled to room temperature and methanesulfonamide (0.13 g, 1.39 mmol) was added and stirred for 10 min. A solution of DBU (0.35 mL, 2.32 mmol) in THF (2.0 mL) was added slowly and stirred at RT overnight under argon. After 24 h, the reaction was found to be complete based on TLC. The reaction completion was confirmed by LCMS. The solvent was removed in vacuo, diluted with sat. aq. NH₄Cl (10 mL) and EtOAc (10 mL). Layers were separated, organic layer was washed with water (2×10 mL), brine (10 mL), dried over Na₂SO₄, filtered, and concentrated in vacuo to obtain crude ralinepag methanesulfonamide (6) (0.33 g). The crude material was purified by silica gel column chromatography. The combined fractions were evaporated in vacuo and dried under high vacuum to give pure ralinepag methanesulfonamide (6) (0.125 g) in 54.3% yield as white solid. The melting point was found to be 117.2-119.2° C. The pure product was characterized by IR, ¹H NMR and LC-MS. HPLC purity of the product was found to be 98.43% and free of any ralinepag acid (1).

### Example 7: Synthesis of Ralinepag Acid Chloride (7)

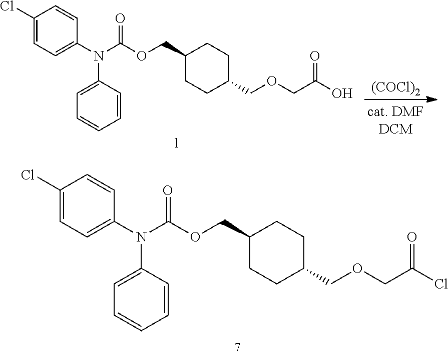

To a stirred solution of ralinepag acid (1) (1.0 g, 2.32 mmol) in DCM (10.0 mL) was added oxalyl chloride (0.60 mL, 6.95 mmol) dropwise at room temperature under argon followed by the addition of catalytic amount of DMF (50 μL) (effervescence formation was observed). The resulting reaction mixture was stirred for 2 h at RT under argon. After 2 h, the reaction was found to be complete based on ¹H NMR. The solvent was removed in vacuo and dried under high vacuum to give pure ralinepag acid chloride (7) (1.06 g) in 100% yield as yellowish solid. The product was characterized by IR, ¹H NMR and LC-MS. The compound is pure enough to be used in next step without further purification.

### Example 8: Synthesis of Ralinepag Glycinamide Methyl Ester (8a)

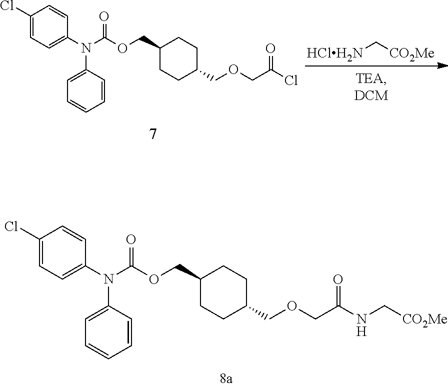

To a stirred solution of ralinepag acid chloride (7) (0.20 g, 0.44 mmol) in DCM (5.0 mL) was added triethylamine (TEA) (0.15 mL, 1.11 mmol) at 0° C. under argon and stirred for 30 min. Then glycine methyl ester hydrochloride (0.08 g, 0.67 mmol) was added and resulting reaction mixture was slowly allowed to warm up to at RT under argon. After 1 h, the reaction was found to be complete based on TLC. The reaction mixture was diluted with DCM (10 mL), washed with water (2×15 mL), brine (10 mL), dried over Na₂SO₄, filtered, and concentrated in vacuo to obtain crude ralinepag glycinamide methyl ester (8a) (0.215 g). This was purified by silica gel chromatography. The combined fractions were evaporated in vacuo and dried under high vacuum to give pure ralinepag glycinamide methyl ester (8a) (0.13 g) in 60.9% yield as white solid. The melting point was found to be 78.3-80.3° C. The pure product was characterized by IR, ¹H NMR and LC-MS. HPLC purity of the product was found to be 99.51% and free of any ralinepag acid (1).

### Example 9: Synthesis of Ralinepag L-Valinamide Methyl Ester (8b)

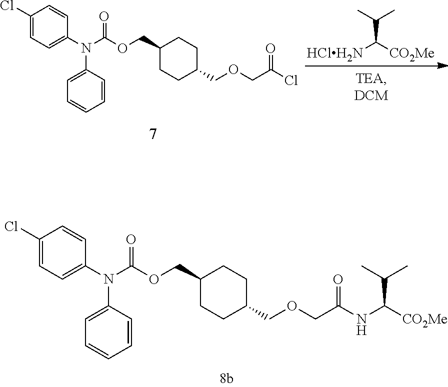

To a stirred solution of ralinepag acid chloride (7) (0.20 g, 0.44 mmol) in DCM (5.0 mL) was added tricthylamine (TEA) (0.15 mL, 1.11 mmol) at 0° C. under argon and stirred for 30 min. Then L-valine methyl ester hydrochloride (0.11 g, 0.67 mmol) was added and resulting reaction mixture was slowly allowed to warm up to at RT under argon. After 2 h, the reaction was found to be complete based on TLC. The reaction mixture was quenched with sat. aq. NH₄Cl (5 mL), extracted with DCM (2×10 mL). Layers were separated and DCM layer was washed with water (10 mL), brine (10 mL), dried over Na₂SO₄, filtered, and concentrated in vacuo to obtain crude ralinepag L-valinamide methyl ester (8b) (0.34 g). This was purified by silica gel chromatography. The combined fractions were evaporated in vacuo and dried under high vacuum to give pure ralinepag L-valinamide methyl ester (8b) (0.19 g) in 77.9% yield as clear viscous oil. The pure product was characterized by IR, ¹H NMR and LC-MS. HPLC purity of the product was found to be 98.20% and free of any ralinepag acid (1).

### Example 10: Synthesis of Ralinepag Bipiperidinamide (8c)

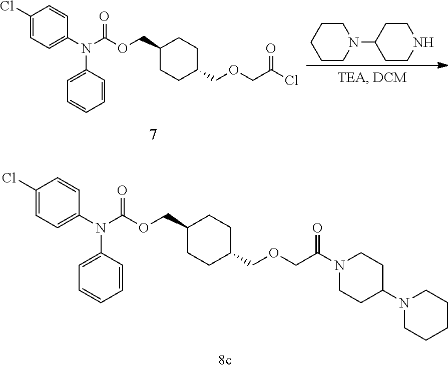 [SMILES: O=C(Cl)COC[C@H]1CC[C@H](COC(=O)N(c2ccccc2)c2ccc(Cl)cc2)CC1 ; C1CCN(C2CCNCC2)CC1 ; O=C(COC[C@H]1CC[C@H](COC(=O)N(c2ccccc2)c2ccc(Cl)cc2)CC1)N1CCC(N2CCCCC2)CC1]

To a stirred solution of ralinepag acid chloride (7) (0.20 g, 0.44 mmol) in DCM (5.0 mL) was added tricthylamine (TEA) (0.15 mL, 1.11 mmol) at 0° C. under argon and stirred for 30 min. Then 4-piperidinopiperidine (0.11 g, 0.67 mmol) was added and resulting reaction mixture was slowly allowed to warm up to at RT under argon. After 1 h, the reaction was found to be ˜90% complete based on TLC. The reaction mixture was quenched with sat. aq. NH₄Cl (5 mL), extracted with DCM (2×15 mL). Layers were separated and DCM layer was washed with water (2×10 mL), brine (10 mL), dried over Na₂SO₄, filtered, and concentrated in vacuo to obtain crude ralinepag bipiperidinamide (8c) (0.30 g). This was purified by chromatography. The combined fractions were evaporated in vacuo and dried under high vacuum to give pure ralinepag bipiperidinamide (8c) (0.17 g) in 67.3% yield as waxy solid. The pure product was characterized by IR, ¹H NMR and LC-MS. HPLC purity of the product was found to be 98.27% and free of any ralinepag acid (1).

### Example 11: Synthesis of Ralinepag Morpholinamide (9)

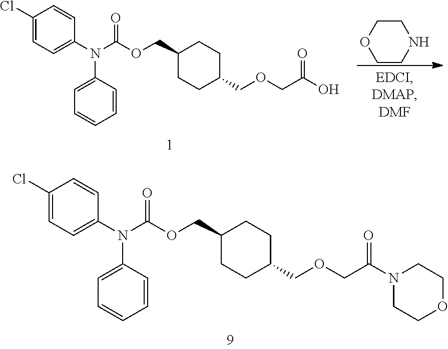 [SMILES: O=C(O)COC[C@H]1CC[C@H](COC(=O)N(c2ccccc2)c2ccc(Cl)cc2)CC1 ; C1COCCN1 ; O=C(COC[C@H]1CC[C@H](COC(=O)N(c2ccccc2)c2ccc(Cl)cc2)CC1)N1CCOCC1]

To a solution of ralinepag acid (1) (0.20 g, 0.46 mmol) in anhydrous DMF (2.5 mL) was added morpholine (0.044 mL, 0.51 mmol) followed by EDCI·HCl (0.13 g, 0.69 mmol) and DMAP (0.17 g, 1.39 mmol) at room temperature under argon. The resulting reaction mixture was stirred overnight. After 19 h, the reaction was found to be complete based on TLC. The reaction mixture was treated with sat. aq. NH₄Cl (2 mL) and extracted with EtOAc (10 mL). The aqueous layer was extracted with EtOAc (2×10 mL) and combined organic layer was washed with water (2×10 mL), brine (10 mL), dried over Na₂SO₄, filtered, and concentrated in vacuo to obtain crude ralinepag morpholinamide (9) (0.34 g). This was purified by silica gel column chromatography. The combined fractions were evaporated in vacuo and dried under high vacuum to give pure ralinepag morpholinamide (9) (0.19 g) in 82.6% yield viscous oil which slowly becomes waxy solid over time. The pure product was characterized by IR, ¹H NMR and LC-MS. HPLC purity of the product was found to be 99.62% and free of any ralinepag acid (1).

What is claimed is:

1. A compound having a structure of Formula (I), or a pharmaceutically acceptable salt or solvate thereof: 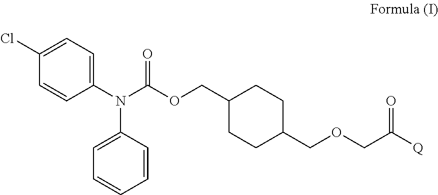
wherein:
Q is —NR⁶—S(═O)₂R⁷;
R⁶ is H, C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
R⁷ is C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L²-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{7a};
L²is absent, C₁-C₆ alkylene, C₁-C₆ heteroalkylene, C₂-C₆ alkenylene, or C₂-C₆ alkynylene, each of which is optionally substituted with one or more R^{7a};
or Q is —OR⁸;
R⁸ is —CH₂CH₂CH₃, —CH(CH₃)₂, —CH₂CH₂CH₂CH₃, —CH₂CH(CH₃)₂, C₅-C₂₄ alkyl, C₁-C₂₄ haloalkyl, C₁-C₂₄ heteroalkyl, C₂-C₂₄ alkenyl, C₂-C₂₄ alkynyl, -L³-O—P(═O)(OH)₂, -L³-cycloalkyl, -L³-heterocycloalkyl, or -L³-aryl, wherein each of the alkyl, heteroalkyl, alkenyl, alkynyl, cycloalkyl, heterocycloalkyl, or aryl is optionally substituted with one or more R^{8a};
L³ is absent, C₁-C₆ alkylene, C₁-C₆ heteroalkylene, C₂-C₆ alkenylene, or C₂-C₂₄ alkynylene, each of which is optionally substituted with one or more R^{8a};
or Q is —NR⁴R⁵;
R⁴ is H, C₁-C₂₄ alkyl, C₁-C₂₄ haloalkyl, C₁-C₂₄ heteroalkyl, C₂-C₂₄ alkenyl, C₂-C₂₄ alkynyl, -L⁵-cycloalkyl, -L⁵-heterocycloalkyl, -L⁵-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{5a};
R⁵ is C₁-C₂₄ alkyl, C₁-C₂₄ haloalkyl, C₁-C₂₄ heteroalkyl, C₂-C₂₄ alkenyl, C₂-C₂₄ alkynyl, -L²-cycloalkyl, -L²-heterocycloalkyl, -L⁵-aryl, or -L²-heteroaryl, wherein each of the alkyl, heteroalkyl, alkenyl, alkynyl, cycloalkyl, heterocycloalkyl, aryl or heteroaryl is optionally substituted with one or more R^{5a}; or
R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a};
L⁵ is absent, C₁-C₆ alkylene, C₁-C₆ heteroalkylene, C₁-C₆ alkenylene, or C₂-C₂₄ alkynylene, each of which is optionally substituted with one or more R^{5a};
R^{5a} and R^{7a} are each independently halogen, —CN, —NO₂, —OH, —OR^{a}, oxo, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{a}, —C(═O)NR^{c}R^{d}, C₁-C₆ alkyl, C₁-C₆ haloalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
R^{8a} is halogen, —CN, —NO₂, —OH, —OR^{a}, oxo, —OC(═O)R^{a}, —SH, —SR^{a}, —S(═O)R^{a}, —S(═O)₂R^{a}, —S(═O)₂OR^{b}, —S(═O)₂NR^{c}R^{d}, —NR^{c}R^{d}, —NR^{b}C(═O)R^{a}, —NR^{b}S(═O)₂R^{a}, —C(═O)R^{a}, —C(═O)OR^{b}, —C(═O)NR^{c}R^{d}, C₁-C₆ alkyl, C₁-C₆ haloalkyl, C₁-C₆ heteroalkyl, C₂-C₆ alkenyl, C₂-C₆alkynyl, cycloalkyl, heterocycloalkyl, aryl, or heteroaryl, wherein each of the alkyl, heteroalkyl, alkenyl, alkynyl, cycloalkyl, heterocycloalkyl, aryl, or heteroaryl is optionally substituted with one or more R;
each R^{a} is independently C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R; or
R^{c} and R^{d} are taken together with the atom to which they are attached to form a heterocycloalkyl optionally substituted with one or more R; and
each R is independently halogen, —CN, —OH, oxo, —OCH₁-C₆alkyl, —S(═O)C₁-C₆alkyl, —S(═O)₂C₁-C₆alkyl, —S(═O)—NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆heteroalkyl;
or Q is: 1-carboxyethylamino, 1-carboxy-4-guanidinobutylamino, 3-amino-1-carboxy-3-oxopropylamino, 1,2-dicarboxyethylamino, 1-carboxy-2-mercaptoethylamino, 4-amino-1-carboxy-4-oxobutylamino, 3-carboxy-1-carboxylatepropylamino, 1-carboxy-2-(1H-imidazol-4-yl)ethylamino, 1-carboxy-2-methylbutylamino, 1-carboxy-3-methylbutylamino, 5-amino-1-carboxypentylamino, 1-carboxy-3-(methylthio)propylamino, 1-carboxy-2-phenylethylamino, 2-carboxypyrrolidin-1-yl, 1-carboxy-2-hydroxyethylamino, 1-carboxy-2-hydroxypropylamino, 1-carboxy-2-(1H-indol-3-yl)ethylamino, 1-carboxy-2-(4-hydroxyphenyl)ethylamino and 1-carboxy-2-methylpropylamino.

2. The compound of claim 1, or a pharmaceutically acceptable salt or solvate thereof, wherein the compound has a structure of Formula (IIIa):

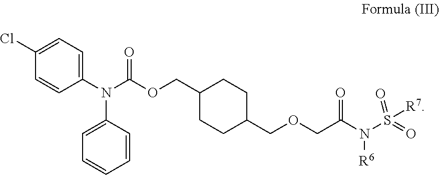

3. The compound of claim 2, or a pharmaceutically acceptable salt or solvate thereof, wherein the compound has a structure of Formula (IIIa):

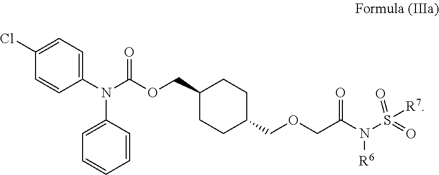

4. The compound of claim 2 or 3, or a pharmaceutically acceptable salt or solvate thereof, wherein R⁶ is H or C₁-C₆ alkyl.

5. The compound of claim 4, or a pharmaceutically acceptable salt or solvate thereof, wherein R⁶ is H.

6. The compound of any one of claims 2 to 5, or a pharmaceutically acceptable salt or solvate thereof, wherein R⁷ is C₁-C₂₄ alkyl, C₁-C₂₄ haloalkyl, or C₁-C₂₄ heteroalkyl, wherein each of the alkyl or heteroalkyl is optionally substituted with one or more R^{7a}.

7. The compound of any one of claims 2 to 6, or a pharmaceutically acceptable salt or solvate thereof, wherein R⁷ is optionally substituted —CH₃, —CH₂CH₃, —CH₂CH₂CH₃, —CH(CH₃)₂, —CH₂CH₂CH₂CH₃, —CH₂CH(CH₃)₂, —CH₂CH₂CH₂CH₂CH₃, or —CH₂CH₂CH₂CH₂CH₂CH₃.

8. The compound of any one of claims 2 to 7, or a pharmaceutically acceptable salt or solvate thereof, wherein R⁷ is methyl,

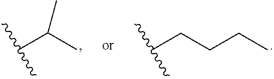

9. The compound of any one of claims 2 to 5, or a pharmaceutically acceptable salt or solvate thereof, wherein:
R² is -L²-cycloalkyl, -L²-heterocycloalkyl, -L²-aryl, or -L²-heteroaryl, wherein each of the cycloalkyl, heterocycloalkyl, aryl or heteroaryl is optionally substituted with one or more R^{7a}; and
R^{7a} is halogen, —CN, —OH, —OR^{a}, oxo, —OC(═O)R^{a}, —SH, —SR^{a}, —S (═O)R^{a}, —S(═O)₂R^{a}, —S(═O)₂NR^{c}R^{d}, —NR^{c}R^{d}, —C(═O)R^{a}, —C(═O)OR^{a}, C₁-C₆ alkyl, C₁-C₆ haloalkyl, or C₁-C₆ heteroalkyl, wherein each of the alkyl or heteroalkyl is optionally substituted with one or more R.

10. The compound of any one of claims 2 to 5, or a pharmaceutically acceptable salt or solvate thereof, wherein:
R⁷ is -L²-phenyl or -L²-naphthyl, wherein each of the phenyl or naphthyl is optionally substituted with one or more R^{7a};
L²is absent, —CH₂—, or —CH₂CH₂—; and
R^{7a} is halogen, oxo, C₁-C₆ alkyl, C₁-C₆ haloalkyl, or C₁-C₆ alkoxy.

11. The compound of any one of claims 2 to 5, or a pharmaceutically acceptable salt or solvate thereof, wherein R⁷ is

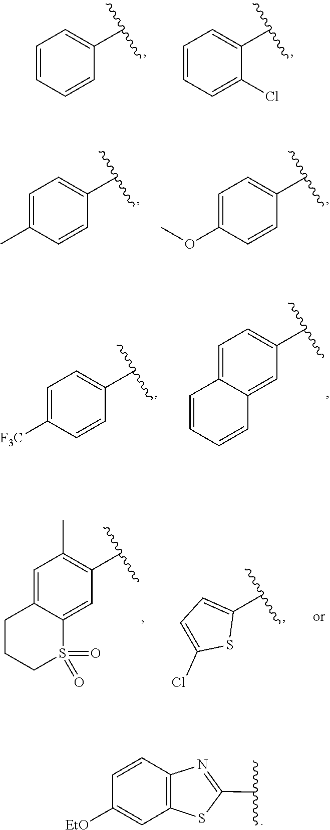

12. The compound of claim 2 or 3, or a pharmaceutically acceptable salt or solvate thereof, wherein

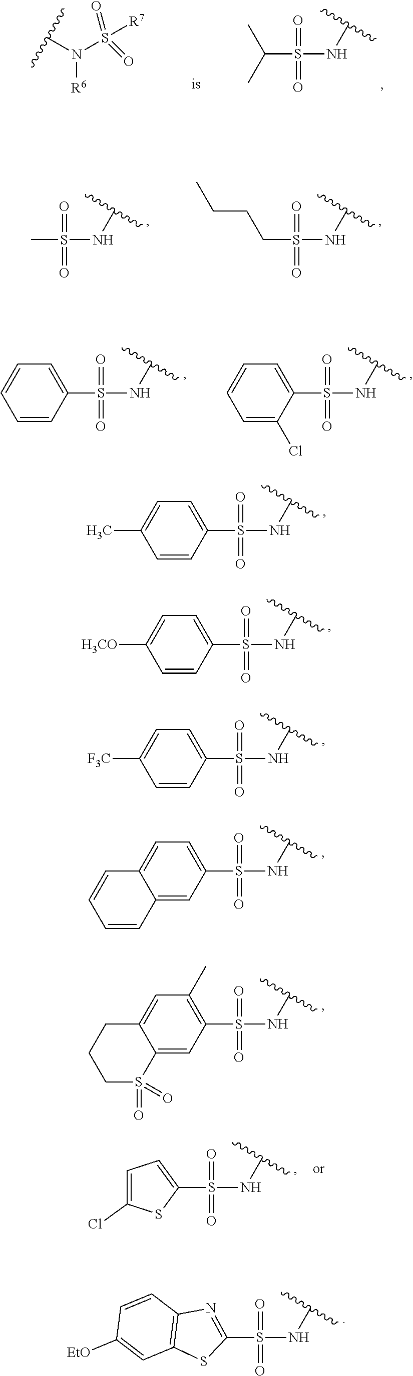

13. The compound of claim 1, or a pharmaceutically acceptable salt or solvate thereof, wherein the compound has a structure of Formula (II)

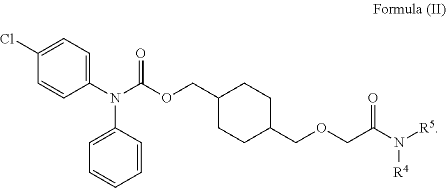

14. The compound of claim 13, or a pharmaceutically acceptable salt or solvate thereof, wherein the compound has a structure of Formula (IIa):

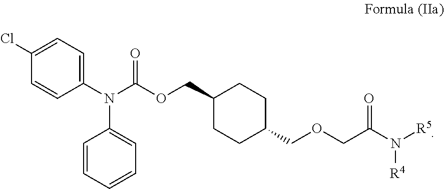

15. The compound of claim 13 or 14, or a pharmaceutically acceptable salt or solvate thereof, wherein R⁴ and R⁵ are taken together with the nitrogen to which they are attached to form a 3 to 12 membered heterocycloalkyl or heteroaryl, wherein each of the heterocycloalkyl or heteroaryl is optionally substituted with one or more R^{5a}.

16. The compound of claim 15, or a pharmaceutically acceptable salt or solvate thereof, wherein R⁴ and R⁵ are taken together with the nitrogen to which they are attached to form a 5 to 6 membered heterocycloalkyl.

17. The compound of any one of claims 13 to 16, or a pharmaceutically acceptable salt or solvate thereof, wherein

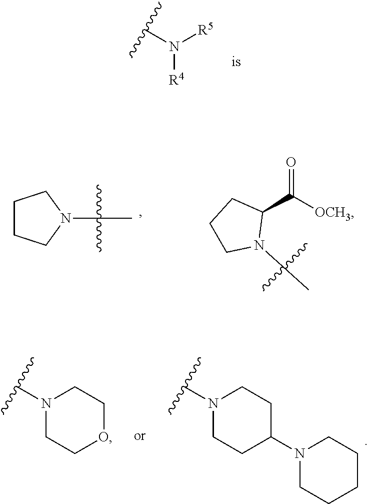

18. The compound of claim 13 or 14, or a pharmaceutically acceptable salt or solvate thereof, wherein R⁴ is H or C₁-C₆ alkyl.

19. The compound of claim 18, or a pharmaceutically acceptable salt or solvate thereof, wherein R⁴ is H.

20. The compound of claim 18, or a pharmaceutically acceptable salt or solvate thereof, wherein R⁴ is C₁-C₆ alkyl.

21. The compound of claim 13 or 14, or a pharmaceutically acceptable salt or solvate thereof, wherein R⁵ is C₁-C₁₂ alkyl, C₁-C₁₂ haloalkyl, C₁-C₁₂ hydroxyalkyl, C₁-C₁₂ aminoalkyl, C₁-C₁₂ heteroalkyl, wherein each of the alkyl or heteroalkyl is optionally substituted with one or more R^{5a}.

22. The compound of claim 21, or a pharmaceutically acceptable salt or solvate thereof, wherein R⁵ is C₁-C₆ alkyl, C₁-C₆ haloalkyl, C₁-C₆ hydroxyalkyl, C₁-C₆ aminoalkyl, C₁-C₆ heteroalkyl, wherein each of the alkyl or heteroalkyl is optionally substituted with one or more R^{5a}.

23. The compound of claim 13 or 14, or a pharmaceutically acceptable salt or solvate thereof, wherein R⁵ is

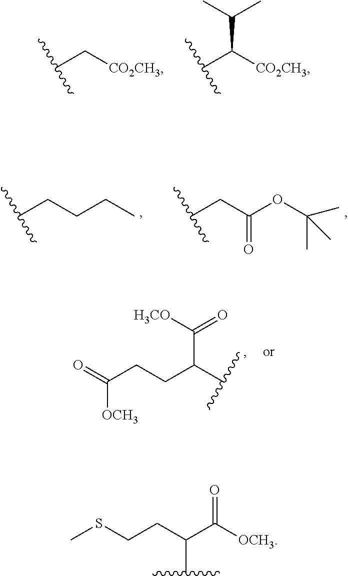

24. The compound of claim 13 or 14, or a pharmaceutically acceptable salt or solvate thereof, wherein R⁵ is -L⁵-cycloalkyl, -L⁵-heterocycloalkyl, -L⁵-aryl, or -L⁵-heteroaryl, wherein each of the cycloalkyl, heterocycloalkyl, aryl or heteroaryl is optionally substituted with one or more substituents selected from halogen, oxo, —OH, C₁-C₆ alkyl, C₁-C₆ haloalkyl, and C₁-C₆ alkoxy.

25. The compound of claim 24, or a pharmaceutically acceptable salt or solvate thereof, wherein L⁵ is C₁-C₆ alkylene or C₁-C₆ heteroalkylene, each of which is optionally substituted with one or more R^{5a}.

26. The compound of claim 13 or 14, or a pharmaceutically acceptable salt or solvate thereof, wherein R⁵ is

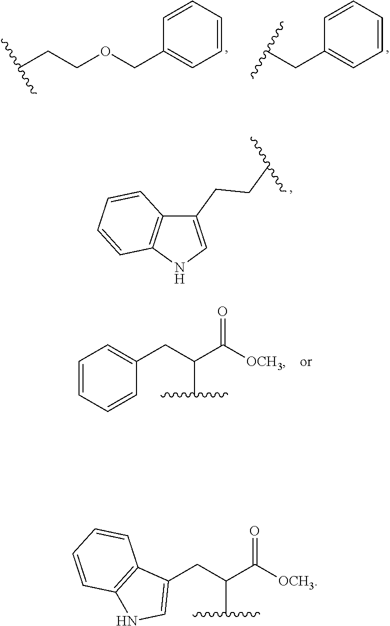

27. The compound of any one of claims 13 to 26, or a pharmaceutically acceptable salt or solvate thereof, wherein R^{5a} is halogen, —CN, —NO₂, —OH, —OR^{a}, oxo, —OC(═O)R³, —OC(═O)OR^{b}, —SH, —SR^{a}, —S(═O)R^{a}, —S(═O)₂R^{a}, —NR^{c}R^{d}, —C(═O)R^{a}, —C(═O)OR^{a}, C₁-C₆ alkyl, C₁-C₆ haloalkyl, C₁-C₆ hydroxyalkyl, C₁-C₆ aminoalkyl, C₁-C₆ heteroalkyl, cycloalkyl, or heterocycloalkyl, wherein each of the alkyl, heteroalkyl, cycloalkyl, or heterocycloalkyl is optionally substituted with one or more R.

28. The compound of any one of claims 13 to 27, or a pharmaceutically acceptable salt or solvate thereof, wherein R^{5a} is halogen, oxo, —C(═O)O—C₁-₃ alkyl, C₁-C₆ alkyl, C₁-C₆ haloalkyl, C₁-C₆ alkoxy, C₃-C₆ cycloalkyl, or C₂-C₅ heterocycloalkyl.

29. The compound of claim 13 or 14, or a pharmaceutically acceptable salt or solvate thereof, wherein

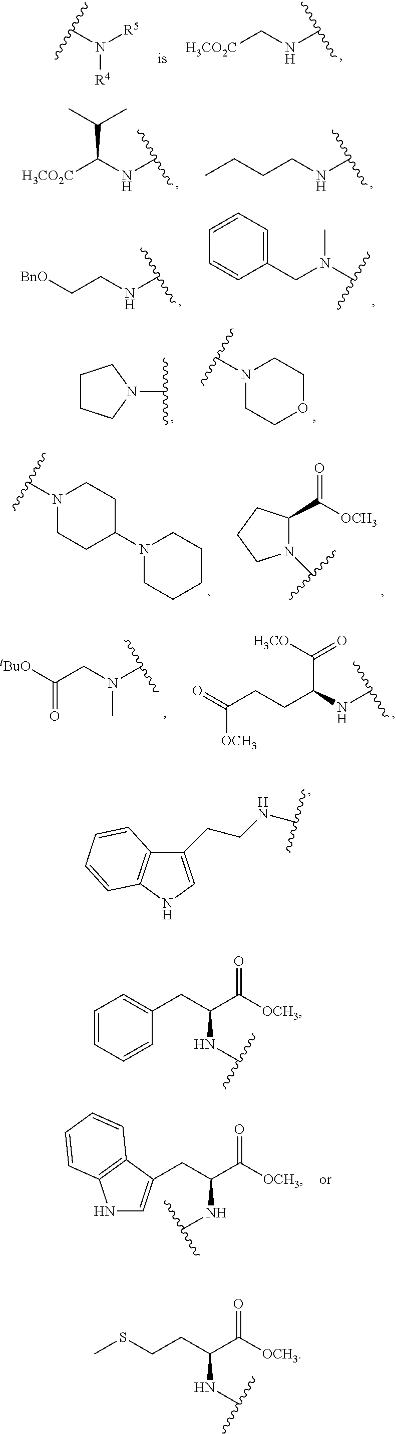

30. The compound of claim 1, or a pharmaceutically acceptable salt or solvate thereof, wherein Q is: (S)-1-carboxyethylamino, (S)-1-carboxy-4-guanidinobutylamino, (S)-3-amino-1-carboxy-3-oxopropylamino, (S)-1,2-dicarboxyethylamino, (S)-1-carboxy-2-mercaptoethylamino, (S)-4-amino-1-carboxy-4-oxobutylamino, (S)-3-carboxy-1-carboxylatepropylamino, (S)-1-carboxy-2-(1H-imidazol-4-yl)ethylamino, (1S,2S)-1-carboxy-2-methylbutylamino, (S)-1-carboxy-3-methylbutylamino, (S)-5-amino-1-carboxypentylamino, (S)-1-carboxy-3-(methylthio)propylamino, (S)-1-carboxy-2-phenylethylamino, (S)-2-carboxypyrrolidin-1-yl, (S)-1-carboxy-2-hydroxyethylamino, (1S,2R)-1-carboxy-2-hydroxypropylamino, (S)-1-carboxy-2-(1H-indol-3-yl)ethylamino, (S)-1-carboxy-2-(4-hydroxyphenyl)ethylamino or (S)-1-carboxy-2-methylpropylamino.

31. The compound of claim 1, or a pharmaceutically acceptable salt or solvate thereof, wherein the compound has a structure of Formula (IV)

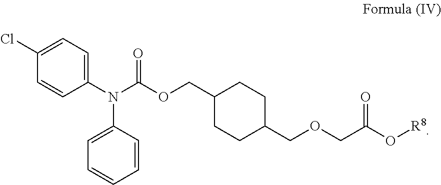

32. The compound of claim 31, or a pharmaceutically acceptable salt or solvate thereof, wherein the compound has a structure of Formula (IVa)

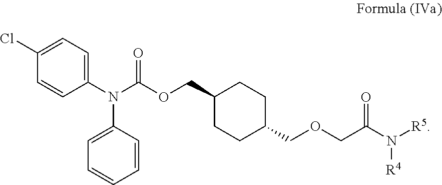

33. The compound of claim 31 or 32, or a pharmaceutically acceptable salt or solvate thereof, wherein R⁸ is —CH₂CH₂CH₃, —CH(CH₃)₂, —CH₂CH₂CH₂CH₃, —CH₂CH(CH₃)₂, C₅-C₁₂ alkyl, C₁-C₁₂ heteroalkyl, C₂-C₁₂ alkenyl, or C₂-C₁₂alkynyl, wherein each of the alkyl, heteroalkyl, alkenyl, or alkynyl is optionally substituted with one or more R^{8a}.

34. The compound of claim 31 or 32, or a pharmaceutically acceptable salt or solvate thereof, wherein R⁸ is

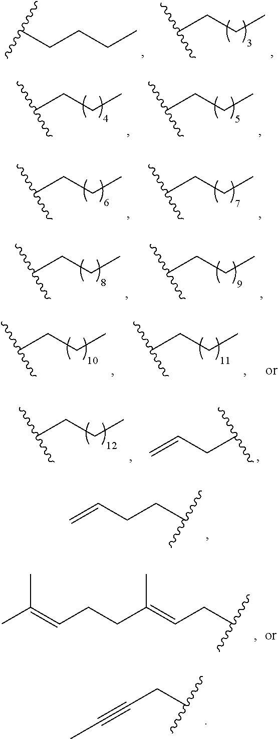

35. The compound of claim 31 or 32, or a pharmaceutically acceptable salt or solvate thereof, wherein R⁸ is

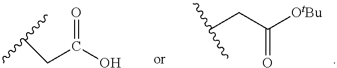 [SMILES: *CC(=O)O ; *CC(=O)OC(C)(C)C]

36. The compound of claim 31 or 32, or a pharmaceutically acceptable salt or solvate thereof, wherein R⁸ is -L³-(5 or 6-membered cycloalkyl), -L³-(5 or 6-membered heterocycloalkyl), —L³-phenyl, -L³-naphthyl, or -L³-heteroaryl, wherein each of the cycloalkyl, heterocycloalkyl, phenyl, naphthyl or heteroaryl is optionally substituted with one or more R^{8a}; L³ is absent, C₂-C₆ alkenylene, C₁-C₆ alkylene or C₁-C₆ heteroalkylene, each of which is optionally substituted.

37. The compound of claim 31 or 32, or a pharmaceutically acceptable salt or solvate thereof, wherein R⁸ is

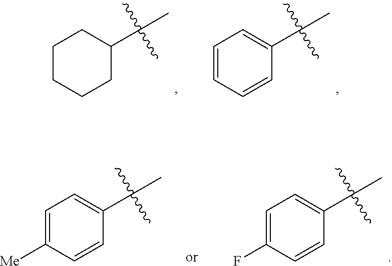 [SMILES: *C1CCCCC1 ; *c1ccccc1 ; *c1ccc(C)cc1 ; *c1ccc(F)cc1]

38. The compound of claim 31 or 32, or a pharmaceutically acceptable salt or solvate thereof, wherein R⁸ is

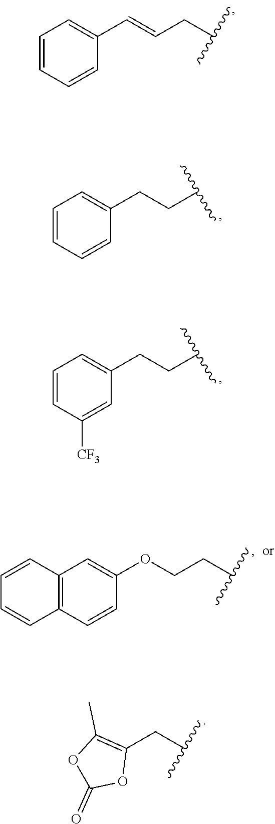

39. The compound of claim 31 or 32, or a pharmaceutically acceptable salt or solvate thereof, wherein R⁸ is -L³-O—P(═O)(OH)₂.

40. The compound of claim 29 or 30, or a pharmaceutically acceptable salt or solvate thereof, wherein R⁸ is

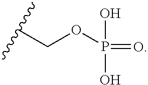 [SMILES: *COP(=O)(O)O]

41. The compound of any one of claims 31 to 40, or a pharmaceutically acceptable salt or solvate thereof, wherein R^{8a} is halogen, —CN, —NO₂, —OH, —OR^{a}, oxo, —OC(═O)R^{a}, —OC(═O)OR^{b}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, C₁-C₆ alkyl, C₁-C₆ haloalkyl, C₁-C₆ heteroalkyl, cycloalkyl, heterocycloalkyl, aryl, or heteroaryl, wherein each of the alkyl, heteroalkyl, cycloalkyl, heterocycloalkyl, aryl, or heteroaryl is optionally substituted with one or more R.

42. The compound of any one of claims 31 to 41, or a pharmaceutically acceptable salt or solvate thereof, wherein R^{8a} is halogen, oxo, C₁-C₆ alkyl, C₁-C₆ haloalkyl, or C₁-C₆ alkoxy.

43. The compound of claim 1, or a pharmaceutically acceptable salt or solvate thereof, wherein the compound is selected from a compound of Table 1.

44. A pharmaceutical composition, comprising a compound of any one of claims 1 to 43, or a pharmaceutically acceptable salt or solvate thereof, and a pharmaceutically acceptable carrier or excipient.

45. The pharmaceutical composition of claim 44, wherein the pharmaceutical composition is in a solid dosage form.

46. The pharmaceutical composition of claim 44 or 45, wherein the pharmaceutical composition is a tablet or a capsule.

47. A method of treating pulmonary arterial hypertension (PAH) in a subject in need thereof. comprising administering to the subject a therapeutically effective amount of a compound of any one of claims 1 to 43, or a pharmaceutically acceptable salt or solvate thereof, or a pharmaceutical composition of any one of claims 44 to 46.

48. The method of claim 47, wherein the subject has one or more World Health Organization (WHO)/New York Heart Association (NYHA) Functional Class (FC) II to III symptoms.

49. The method of claim 47 or 48, wherein the PAH is selected from:
idiopathic PAH;
familial PAH;
PAH associated with a collagen vascular disease selected from: scleroderma, CREST syndrome, systemic lupus erythematosus (SLE), rheumatoid arthritis, Takayasu's arteritis, polymyositis, and dermatomyositis;
PAH associated with a congenital heart disease selected from: atrial septic defect (ASD), ventricular septic defect (VSD) and patent ductus arteriosus in an individual;
PAH associated with portal hypertension;
PAH associated with HIV infection;
PAH associated with ingestion of a drug or toxin;
PAH associated with hereditary hemorrhagic telangiectasia;
PAH associated with splenectomy;
PAH associated with significant venous or capillary involvement;
PAH associated with pulmonary veno-occlusive disease (PVOD); and
PAH associated with pulmonary capillary hemangiomatosis (PCH) in an individual.

50. The method of claim 47 or 48, wherein the PAH is familial primary pulmonary hypertension.

51. A method of modulating a prostacyclin (PGI2) receptor in a subject in need thereof, comprising administering to the subject a therapeutically effective amount of a compound of any one of claims 1 to 43, or a pharmaceutically acceptable salt or solvate thereof, or a pharmaceutical composition of any one of claims 44 to 46.

52. A method of treating a disease or condition associated with a prostacyclin (PGI2) receptor in a subject in need thereof, comprising administering to the subject a therapeutically effective amount of a compound of any one of claims 1 to 43, or a pharmaceutically acceptable salt or solvate thereof, or a pharmaceutical composition of any one of claims 44 to 46.

53. The method claim 52, wherein the disease or condition is selected from: pulmonary arterial hypertension (PAH), pulmonary hypertension, hypertension, connective tissue diseases, vascular diseases, cardiovascular diseases, lung diseases, and respiratory tract disease.

54. The method of any one of claims 47 to 53, wherein the compound is administered via a titration scheme.

55. The method of any one of claims 47 to 54, wherein the compound is administered once daily.

56. The method of any one of claims 47 to 54, wherein the compound is administered twice daily.

57. The method of any one of claims 47 to 56, wherein the compound is administered in an amount of about 0.01 mg to about 2 mg per day.

58. The method of any one of claims 47 to 56, wherein the compound is administered in an amount of about 0.05 mg to about 1.2 mg per day.

59. Use of a compound of any one of claims 1 to 43, or a pharmaceutically acceptable salt or solvate thereof, or a pharmaceutical composition of any one of claims 44 to 46, in the manufacture of a medicament for the treatment of pulmonary arterial hypertension (PAH).
